(19) United States
(12) Patent Application Publication
(10) Pub. No.: US 2025/0093845 A1
(43) Pub. Date: Mar. 20, 2025

(54) SYSTEMS FOR CONTROLLING DESIGN AND MANUFACTURING PROCESS FOR USER SPECIFIC DEVICES

(71) Applicant: 3D LIFEPRINTS UK LTD., Liverpool (GB)

(72) Inventors: Matthew GAILER, London (GB); Henry PINCHBECK, London (GB); Paul FOTHERINGHAM, London (GB)

(73) Assignee: 3D LIFEPRINTS UK LTD., Liverpool (GB)

(21) Appl. No.: 18/424,948

(22) PCT Filed: Oct. 11, 2021
(86) PCT No.: PCT/EP2021/078024

§ 371 Date: Jan. 29, 2024

(51) Int. Cl. G05B 19/4097

(52) U.S. Cl. CPC G05B 19/4097; G05B 2219/35217

(57) ABSTRACT

Described herein are systems and methods for controlling a design and manufacturing process of a user-specific device (e.g., a patient-specific medical device). A method involves receiving, from the requesting entity user-specific information related to a requested device. Based on the information, a list of devices matching user-specific information is determined and displayed for selection by the requesting entity. Based on the information and the selection, a sequence of tasks for design and manufacturing of the user-specific device is generated. Based on the received information and the tasks, a designing entity can generate an interactive digital model of the user-specific device. Based on the interactive model, feedback is received from the requesting entity. Based on the feedback, the interactive digital model and sequence of tasks may be updated. Depending on a location of the requesting entity, the sequence of tasks or devices can be configured to satisfy regulatory compliance.

### FIELD OF THE INVENTION

The present disclosure relates to device manufacturing process of user-specific devices, e.g., medical devices. More particularly, controlling one or more aspects of the design and manufacturing process by reference to user-specific requirements and characteristics.

### BACKGROUND OF THE INVENTION

Manufacturing of devices is typically a standardized process where a device is designed and based on that single design, appropriate manufacturing steps are defined. A standard device is generally mass manufactured which requires the design and manufacturing process be fixed with little room for either design or manufacturing set-up changes. However, for certain user-specific devices (e.g., patient-specific medical devices) there is a requirement that each device is designed and manufactured individually based upon the user characteristics and requirements. As such, user-specific devices cannot be manufacturing using the existing mass manufacturing process. The advent of commercial scale three-dimensional (3D) printing has opened up the potential for mass manufacturing of user-specific devices but this opportunity requires controls to be put in place for both the design and manufacturing processes to ensure the devices consistently meet the user characteristics and requirements as well as relevant quality and regulatory specifications.

### SUMMARY OF THE INVENTION

Aspects of the invention relate to methods, apparatuses, and/or systems for controlling end-to-end design and manufacturing process of user-specific devices. In an embodiment, the controlling may be achieved by integrating data exchange and design/manufacturing processes between the different entities involved in the process. For example, the different entities may be a subject entity (e.g., a patient for whom a medical device is created), a requesting entity (e.g., a medical professional such as a surgeon requesting a device for a patient), a designing entity (e.g., engineer or designer of devices), a manufacturing entity (e.g., manufacturer of the devices). Design and manufacture of user-specific devices can be complex as the structure of body parts of the subject entity creates a unique environment in which the device may be deployed. For example, a user-specific medical device associated with an individual patient should have specific characteristics corresponding to that patient's unique anatomy. According to the present disclosure, controlling mechanisms herein ensure that a user-specific device is designed in a certain way before manufacturing of the user-specific device. For example, the mechanisms herein enable the device to be appropriate for the environment (e.g., patient's anatomy), and modified to conform to the shape of that environment before manufacturing the user-specific device. Such structural modification may also cause changes to the manufacturing process.

According to an embodiment of the present disclosure, the mechanism for controlling the end-to-end process of design and manufacturing of user specific devices includes cloud-based servers and databases accessible via a technology platform that enables secure exchange of user-specific information and designs between the different entities. The technology platform herein is the framework for exchanging information and algorithms contained within the platform serve as a means to enable the selection of appropriate devices and their characteristics for a specific request from a user.

In an embodiment, a user interface section of the platform facilitates; dynamic generation of user-specific requirements, displaying and manipulating digital models of the device designs, receiving feedback on the designs, and displaying the updated designs based on the feedback. In an embodiment, the user specific requirements captured by the user interface determine a sequence of tasks that controls the design and manufacturing process of each user-specific device (e.g., medical devices).

According to an aspect of the present disclosure, a system for controlling the design and manufacturing process of a user specific device. The system includes a computer system that comprises one or more processors programmed with computer program instructions that, when executed, cause a set of operations. The operations include receiving, via a graphical user interface, from a requesting entity, (i) information related to a device to be manufactured to conform to a subject entity, (ii) a selection by the requesting entity of one or more devices from a list of devices, wherein the information comprises: a type of application, structure of a body part of the subject entity, an intended use of a device, and intended outcome upon use of a device; generating, based on the received information and the selection of the one or more devices, a sequence of tasks for design and manufacturing of the user-specific device; generating an interactive digital model of the user-specific device, the interactive digital model being generated by a designing entity based on the received information and within the sequence of tasks generated by the system; transmitting the interactive digital model associated with the user-specific device to the graphical user interface, the graphical user interface being configured to allow the requesting entity to interact with the interactive digital model; receiving, via the graphical user interface, feedback from the requesting entity on the interactive digital model, the feedback comprising errors or modifications associated with the user-specific device; and updating, based on the feedback, the interactive digital model by the designing entity and the sequence of tasks to incorporate the feedback and design changes by the designing entity.

In an embodiment, the list of devices includes one or more of: an anatomical model(s) of the part of the subject entity; a surgical guide(s) to be used during surgery; an implant(s) or other devices.

In an embodiment, the information provided or derived based on inputs from the requesting entity may include device characteristics. For example, the device characteristics include a particular material based on the intended use of the device; a customized geometry to fit the structure of the subject entity's body part; a sub-component or a particular area of interest of the structure of the subject entity's body part; or a combination thereof. As an example, the particular material for the device has one or more material property comprising: biocompatibility, flexibility, durability, transparency, utility and/or life-like appearance.

In an embodiment, the intended use of the device comprises at least one of: a pre- or an intra-surgical planning; visual communications; surgical simulation prior to an intended procedure; an implantation in the subject entity; or other purposes for which medical devices may be used.

In an embodiment, the list of devices may be determined based on the type of application. In an embodiment, the list of devices determined based on the selected one or more devices and the received information includes suggested complementary or alternative devices to the user-specific device selected by the requesting entity.

In an embodiment, generating the sequence of tasks involves selecting or updating, based on the information provided by the requesting entity, the sequence of tasks from a workflow database corresponding to the device characteristics, the intended use of the device, an acceptable quality specification, or regulation specifications associated with the user-specific device.

In an embodiment, generating the interactive digital model of the device involves determining, by the designing entity, a location and an orientation within the subject entity's environment, a geometry of the user-specific device based on the information provided by the requesting entity, and a material of the user-specific device compatible with the intended use of the device.

In an embodiment, receiving the feedback from the requesting entity on the interactive digital model involves: causing the graphical user interface to display and manipulate the interactive digital model of the user-specific device, or the environment of the subject entity adjacent to each other or superimposed on each other in a first portion of the graphical user interface; causing the graphical user interface to annotate and/or highlight one or more portions of the interactive digital model of the user-specific device; causing the graphical user interface to display an editable comments box in a second portion of the graphical user interface, the second portion being adjacent to the first portion; and receiving, from the requesting entity, a manipulation, an annotation, a highlight, and/or, a comment associated with the interactive digital model. In an embodiment, the requesting entity provides a confirmation that the interactive digital model is approved; and the approval is transmitted to the designing entity.

In an embodiment, the operations further involves transmitting the feedback on the interactive digital model associated with the user-specific device to the designing entity; changing based on the feedback, the design and corresponding interactive digital model associated with the user specific device by the designing entity, and/or the manufacturing process of the user-specific device; and reloading, on the graphical user interface, the updated interactive digital model for further comments or approval by the requesting entity. In an embodiment, changing the interactive digital model associated with the user-specific device involves: analyzing the feedback to determine changes to the interactive digital model; and updating resources suggested for the manufacturing process of the user-specific device.

In an embodiment, the operations further involve allocating, based on the information, and/or the feedback from the requesting entity, one or more resources for the design and/or manufacturing of the user-specific device based on capability and capacity of the one or more resources, the allocated resource comprising the designing entity and a manufacturing entity. In an embodiment, allocating the one or more resources involves: predicting an amount of time and a number of personnel required for designing and/or manufacturing of the user-specific device; predicting a total cost of the design and manufacture of the user-specific device; and determining, based on the time and cost, whether additional resources than the designing and manufacturing entity are required within the sequence of tasks to meet any deadline for supply of the user-specific device. In an embodiment, allocating the one or more resources comprises: distributing of the device design, the interactive digital model or the updated interactive digital model associated with the user-specific device to one or more personnel across distributed sites; and/or distributing of the manufacturing of the user-specific device to one or more distributed sites. In an embodiment, allocating the one or more resources involve assigning manufacturing resources based on a geographical location of the requesting entity, the capability and capacity associated with one or more available resources of the designing and manufacturing entities.

In an embodiment, the operations further involve generating documentations for regulatory compliance. For example, a geographical location is received as part of the information received from the requesting entity. Based on the geographical location, relevant jurisdiction associated with the geographical location, regulatory specifications and/or quality assurance constraints related to a user-specific device for that jurisdiction is determined. Then, the sequence of tasks is updated for the designing and manufacturing entities to comply with the requirements of the jurisdiction, and regulatory and quality assurance documentations is generated to meet the requirements of the jurisdiction.

According to an aspect of the present disclosure, a computer-implemented graphical user interface is provided. In an embodiment, a computer-implemented method for dynamically updating a graphical user interface for controlling design and manufacturing processes for user-specific devices is provided. The method involves receiving, via a graphical user interface, a first set of information related to a user-specific device from a requesting entity, the first set of information comprises a type of application, structure of a body part, an intended use of the user-specific device, and intended outcome upon use of the user-specific device; generating a second set of information based on the first set of information, the second set of information comprising a list of devices and device characteristics based on the first set of information related to the user-specific device; receiving, via the graphical user interface, a selection of one or more devices from the list of devices from the requesting entity; generating a set of questions associated with the selected devices to capture additional user-specific information for designing and/or manufacturing the user-specific device; receiving, via the graphical user interface, responses to the set of questions; receiving, from a designing entity, an interactive digital model associated with the user-specific device and/or a manufacturing process for manufacturing the user-specific device, the interactive digital model being generated based on the selected devices and responses to the set of questions; prompting, via the graphical user interface, the requesting entity to provide feedback on the interactive digital model, the feedback comprising errors or modifications associated with the interactive digital model; and updating, based on the feedback, the graphical user interface to display an updated interactive digital model associated with the device for approval by the requesting entity.

According to an aspect of the present disclosure, there is provided, a system for controlling design and manufacturing processes of a medical device for a patient. The system includes a computer system that comprises one or more processors programmed with computer program instructions that, when executed, cause operations involving receiving, via a graphical user interface, patient-specific information related to a medical procedure to be performed by a requesting entity, the patient-specific information comprises a type of application, anatomy of a subject entity, an intended use of the medical device, and intended outcome upon use of the medical device; determining a list of medical devices and device characteristics based on the patient-specific information related to the medical procedure; responsive to selection of one or more medical devices from the list of medical devices by the requesting entity, determining additional patient-specific information to be requested from the requesting entity; generating, based on the received information and the selection of the one or more devices, a sequence of tasks for design and manufacturing of the patient-specific device; generating an interactive digital model for a patient-specific medical device and/or a manufacturing process for manufacturing the patient-specific medical device, the interactive digital model being generated by a designing entity based on the received information and the sequence of tasks; transmitting the interactive digital model of the patient-specific medical device to the graphical user interface, the graphical user interface being configured to allow the requesting entity to interact with the interactive digital model; receiving, via the graphical user interface, feedback from the requesting entity on the interactive digital model, the feedback comprising errors or modifications associated with the patient-specific medical device; and updating, based on the feedback, the interactive digital model by the designing entity and the sequence of tasks to incorporate the feedback and design changes by the designing entity.

Various other aspects, features, and advantages of the invention will be apparent through the detailed description of the invention and the drawings attached hereto. It is also to be understood that both the foregoing general description and the following detailed description are examples and not restrictive of the scope of the invention. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

FIG. 1 is an overview block diagram illustrating a system used to an exchange of information and feedback between different entities to enable controlling of a manufacturing process of a user-specific device, in accordance with one or more embodiments.

FIG. 2 is a schematic illustration of a system configured for controlling a manufacturing process of user-specific devices, in accordance with one or more embodiments.

FIG. 3 is an exemplary flow chart of an algorithm used to control the end-to-end design and manufacturing process of a user-specific device, in accordance with one or more embodiments.

FIG. 4 illustrates an exemplary dynamic electronic exchange form, in accordance with one or more embodiments.

FIGS. 5A-5C illustrate exemplary medical devices displayed on a graphical user interface, in accordance with one or more embodiments.

FIG. 6 is an example of patient-specific data represented as a volumetric scan of a body part (e.g., a head), the scan data including a 3D rendering, a top view, a front view, and a side view of the body part (e.g., the head), in accordance with one or more embodiments.

FIG. 7 illustrates an exemplary feedback and approval process of a first medical device achieved via a graphical user interface, in accordance with one or more embodiments.

FIG. 8 illustrates an exemplary feedback and approval process of a second medical device achieved via the graphical user interface, in accordance with one or more embodiments.

FIG. 9 is an exemplary flowchart of a method of controlling end-to-end design and manufacturing process of devices, in accordance with one or more embodiments.

FIG. 10 is an exemplary flowchart of a method of controlling end-to-end manufacturing process of devices, in accordance with one or more embodiments.

### DETAILED DESCRIPTION OF THE INVENTION

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

Manufacturing user-specific devices can be challenging and time-consuming process. For example, manufacturing of user-specific devices such as a medical device is constrained by the fact that each device requires certain unique characteristics. Unique constraints may arise, for example, because a structure of a body part of a first patient may be substantially different from a structure of the same body part of another patient. Hence, a device for the first patient may not be readily used for the second patient. Additional constraints may arise due to treatment procedure to be employed by a particular expert (e.g., a surgeon), for example, during implanting the device in the patient. These constraints create issues when manufacturing the devices within a design and manufacturing system that satisfy quality assurance and regulatory compliance. The commercialization of user-specific devices is also hampered by the inability of a manufacturer to predict timelines for delivery or calculate margin. As such improved mechanisms are needed for controlling the design and manufacture of user-specific devices within the constraints associated with the devices.

In the present disclosure, the term “device design,” or “designing” refers to structural and functional aspects of the device. For example, the structural aspect include, but not limited to, shape, size, material, density, transparency, or other structural characteristics of the device. The functional aspect refers to the intended use, and operation of the device. Accordingly, different designs of a device may refer to different structures or functions the devices may perform based on user-specific requirements. The term “workflow” or “sequence of tasks” refers to a set of activities or tasks to be performed by a computer system, and/or one or more entities (e.g., requesting entity, designing entity, manufacturing entity, etc.) involved in device request and its design and manufacturing so that a requested device is designed and manufactured according to the user-specific requirements and within a desired time period. Each task may be associated with a trigger (e.g., event-based or time-based), an action, and/or a time period within which the action is desired to be completed. The sequence of tasks may vary depending on the user-specific requirements, device design, or other factors influencing the manufacturing of a requested device. In some embodiments, one or more tasks may be performed simultaneously. In some embodiments, a particular task may be performed in a sequence.

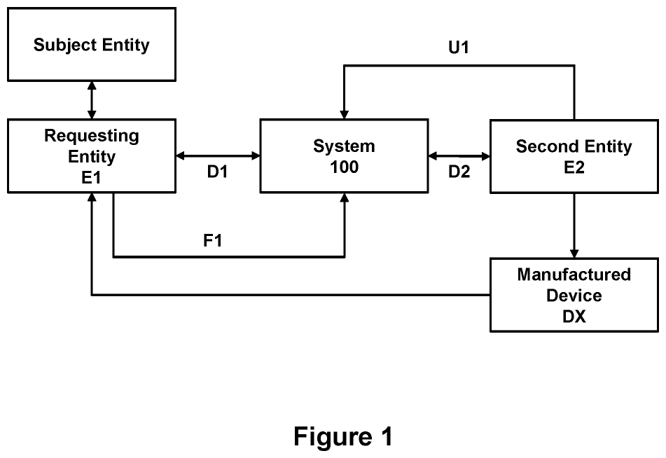

FIG. 1 is a block diagram illustrating data exchange and interactions between different entities via system 100 for controlling a user-specific device manufacturing process, in accordance with one or more embodiments. In an embodiment, the user-specific device manufacturing process may be an end-to-end manufacturing process including pre-manufacturing, manufacturing, post-manufacturing stages, or a combination thereof. For example, the stages include, but not limited to, conception, design, design optimization, review, feedback, manufacturing, and delivery stage. As shown in FIG. 1, the system 100 controls one or more stages of the user-specific device manufacturing process by presenting potential solutions related to desired outcomes to a requesting entity so that appropriate manufacturing related information may be collected for designing the user-specific device and for defining a manufacturing flow for the user-specific device. Based on the user-specific needs and solutions, different manufacturing information and manufacturing flows may be generated so that different devices may be customized to address user-specific needs and issues.

In an embodiment, the system 100 may be configured to perform a regional configuration. For example, a requesting entity's country details may be stored in a customer database. So, when a new device request is received the system may preconfigure a workflow for any regulatory specification in the corresponding requesting entity's territory.

In an embodiment, the system 100 may perform a case type or model selection. In an embodiment, when the requesting entity selects a type of case from a new device request form, the system automatically determines a number and type of devices for completing the request. For example, for a new request for a craniomaxillofacial (CMF) implant, the system 100 may generate or suggest three devices—an anatomical model for diagnostics, a surgical cutting guide for intra-surgical purposes and the implant itself.

In an embodiment, the system 100 may be configured to determine manufacturing information for the new device request. In an embodiment, the system 100 matches a new device request with a 3D printer, a material, or both. In an embodiment, the system 100 comprises a rules database having a list of rules based on a type of procedure and a nature of the device requested. The rules are used to determine a preferred manufacturing technology (e.g., 3D print technology and raw material) to achieve a suitable product for the procedure involved. In some cases, manufacturing is subcontracted for particular products and such subcontracting information is also provided and tracked as a part of the workflow, and the workflow updated accordingly.

In an embodiment, the system 100 may be configured to perform resource selection based on the requested device. For example, one or more engineers may be assigned to the requesting entity in the customer database. The engineer's workload and customer assignments may be tracked within the system 100. The system 100 may include an algorithm for resource allocation, where the algorithm may select and assign a most appropriately qualified engineer with suitable capacity to the newly request device, and the engineer may be notified about the assignment by email.

In an embodiment, the system 100 may be configured to determine a workflow selection based on information related to the requested device. For example, workflows for each device category with a series of deliverables to be tracked are stored within a project template database. As a device type is selected, the corresponding workflow is automatically assigned in the system 100. This ensures that engineers time is controlled appropriately on each project (e.g., for use with capacity management) and also that any corresponding regulatory specifications assigned based on the territory can be outlined and monitored in the same workflow.

In an embodiment, the system 100 selects one or more devices based on data received from the requesting entity. For example, a use case (e.g., an intended use) identified in a device request form (from a list of pre-determined device types) may be used to match a set of rules stored within a project database. A logic is then applied to a new project to assign a status (e.g., a patient specific medical device, educational device, etc.) to the device. This selection step is important because although the inputs and outputs of a project can be similar the use case can determine whether the requested device is used for diagnostic or treatment purposes. Based on the purpose, the requested device may be classified as a user-specific device as opposed to an educational or research purpose device which would exempt the requested device from the medical device classification.

In an embodiment, the system 100 facilitates dynamic generation and receiving of user-specific device information D1 from a requesting entity E1 to conform to requirements of a subject entity, determining manufacturing related information D2 based on the user-specific information D1, and augmenting the manufacturing decisions with the user-specific device information D1. The system 100 may transmit the device information D1 to a second entity E2 for designing and/or determining manufacturing steps. As an example, the second entity E2 may be a designing entity that determines the shape, size, material, etc. based on the information D1 received from the requesting entity E1. As another example, the second entity E2 may be a manufacturing entity that determines the manufacturing workflow based on the design of the user device received from the designing entity and information received from the requesting entity E1. The system 100 may determine manufacturing related information D2 from databases and/or the second entity E2 that may be translated into questions for the requesting entity E1 to dynamically collect use-specific information.

For example, the device information D1 may include intended use and desired outcome based on which potential list of devices may be determined that achieve the desired outcome. The system 100 further receives feedback information F1 from the requesting entity E1 and based on the feedback information F1, the second entity E2 updates the design/manufacturing related information D2 to generate the updated information U1. Once, the updated information U1 is approved by the requesting entity E1, the second entity may manufacture the user-specific device DX and ship it to the requesting entity E1.

In an embodiment, the system 100 for controlling design and manufacturing of the user-specific device may be configured to prompt the requesting entity E1 to provide appropriate user-specific information (e.g., a desired outcome, a type of application, etc.), and suggest a list of devices and their characteristics based on the user-specific information that can potentially solve the user issue. The system 100 implements an algorithm configured to determine the list of devices, and/or manufacturing flow or steps based on information (e.g., intended use or desired outcome) in the user-specific information. The algorithm comprises several decision points that lead to selecting one device design over another, selecting one manufacturing flow over another, or other functions based on the user-specific information. In an embodiment, one or more predictive models may be executed to predict estimated delivery times, cost, resources, etc. The predictive model also enables updating of the delivery times, resource allocation, and costs for any changes that may occur based on the feedback from the requesting entity. In an embodiment, the model may be a linear model, quadratic model, or other mathematical models.

In an embodiment, the requesting entity E1 may be any person with knowledge about the structure and functions of the body parts (e.g., anatomy of human body parts), procedures required to implant a device in the body part, pre-planning required to implant the device, devices that may be employed to fix the issues related to the body part, or having other information related to the device and a user. For example, the requesting entity may be a healthcare professional such as doctors, surgeons, clinicians, or others who can provide information about anatomy of a subject entity receiving a treatment or a medical device. In an embodiment, the second entity E2 is different from the requesting entity E1. The second entity E2 may be any person involved in designing and/or manufacturing of the device based on the inputs from the requesting entity E1. For example, the second entity E2 may be engineers, designers, regulatory compliance officers, production manager, or other personnel related to design, manufacturing and delivery of devices.

In some embodiments, based on the user-specific inputs (e.g., structural and procedural constraints), system 100 generates an interactive digital model associated with one or more user-specific devices prior to manufacturing for review by the requesting entity. Since, the user-specific device may depend on a structure of the body part, the review of the interactive digital model of the device provides a visual guidance to the requesting entity on how the device will interact or correspond to the structure of the body part (e.g., anatomy of human body part). Based on the interactive digital model, the requesting entity may determine whether the device will provide desired results to the user upon using the device in reality. Such determination may not be done by the second entity since the second entity may not have knowledge about how to analyse impact of changes to the design, and whether desired results may be obtained due to such changes. Upon review, the requesting entity may manipulate or provide feedback on the interactive digital model to accurately point out the changes, if any, as needed. In this way, the interactive digital model advantageously captures additional user-specific data (e.g., structural and procedural constraints) and incorporates it directly on the device design.

When such feedback is transmitted to the second entity, changes to the device design can be implemented according to user-specific requirements. Advantageously, providing and accessing the feedback changes directly from the interactive digital model provides enables the system to transmit user-specific data to the second entity, as well as make changes to design/manufacturing workflow (e.g., a sequence of tasks to be performed by one or more entities at a given point in time) corresponding to the feedback changes. Such visual guidance is beneficial to understand and incorporate the requested changes in the device design, the manufacturing process, or both. Thus, the system enables generation of the manufactured device (e.g., DX in FIG. 1) having a structure and functions that are user-specific. Accordingly, the manufactured device will be more accurate and satisfy user-specific needs (structurally and functionally) compared to a mass manufactured device. Also, waste or remanufacturing of a user-specific device can be prevented.

Another advantage of the system 100 is that it improves inter process communication more efficient by accessing and sharing information based on user-specific data from different databases such as databases configured with user-specific information, device information, manufacturing information, or other information stored in different databases. The system 100 facilitates generating an improved solution (e.g., in terms of devices, procedures and manufacturing process) by making the interaction between the requesting entity and the second entity more efficient resulting in a more efficient overall manufacturing process. For example, timely feedback and changes ensure timely manufacture and delivery of medical devices to the requesting entity so that a subject entity such as a patient may receive a desired treatment in a reasonable time. In an embodiment, efficient interactions refer to less number of interactions facilitated via dynamic input generation to capture most relevant user-specific information, rather than generic information that may require several follow-up questions from the second entity. In an embodiment, efficient interaction refers to having all the information about the overall manufacturing process accessible via a single client portal of the technology platform, rather than logging into different portal to securely access desired information about requested devices. For example, in existing systems, the requesting entity may need to correspond with the designing entity or the manufacturing entity over emails several times, open different files on different software or login-based portals, track the delivery from a different portal, etc. This leads to highly inefficient process wasting valuable time of the requesting entity (e.g., doctors, surgeons). Also, the user-information may not get communicated in an effective manner to the designing entity of the manufacturing entity.

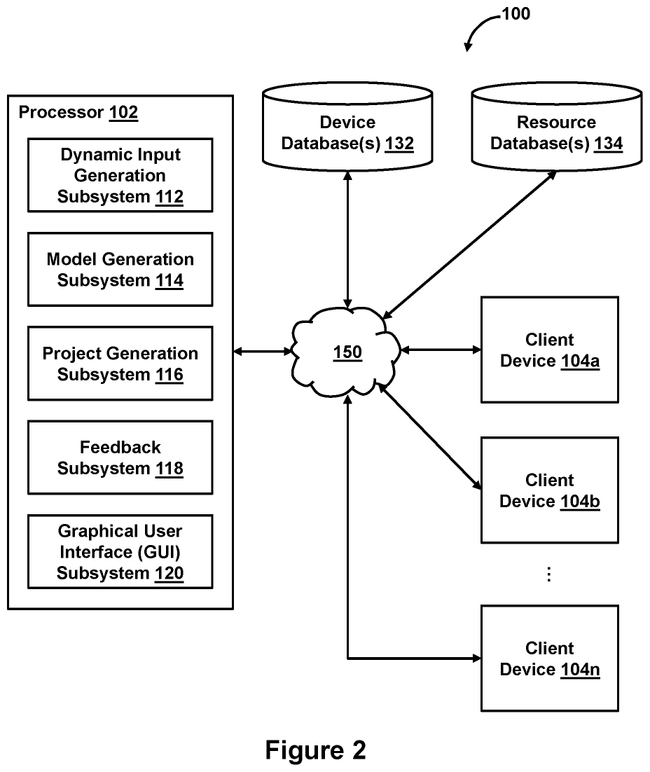

In an embodiment, as shown in FIG. 2, the system 100 may include processor 102, client device 104 (or client devices 104a-104n), device database 132, resource database 134, or other components. In an embodiment, the client device 104a may be accessed by one or more requesting entities (e.g., surgeons, clinicians, etc.) that are requesting one or more user-specific devices for a user. The one or more requesting entities are knowledge about analyzing user problems and solutions of a subject entity. For example, the requesting entity can understand and analyze implementation and functioning of user-specific devices in a given environment (e.g., anatomy of the subject entity), or operating procedures using the user-specific devices. In an embodiment, the requesting entity may be located in a first location and not knowledgeable about the design and manufacturing processes of the one or more user-specific devices. In an embodiment, another client device 104b may be accessed by one or more second entities (e.g., CAD designers, engineers, manufacturing experts, tool suppliers, etc.) that are in charge of designing, manufacturing, or both of the one or more user-specific devices. The second entity may be located at a second location and not knowledgeable about analyzing user problems (e.g., treatment, fracture, disease, etc.). As such, the second entity may not be able to analyze how any changes to the user-specific device may improve the solution or negatively impact the user problems.

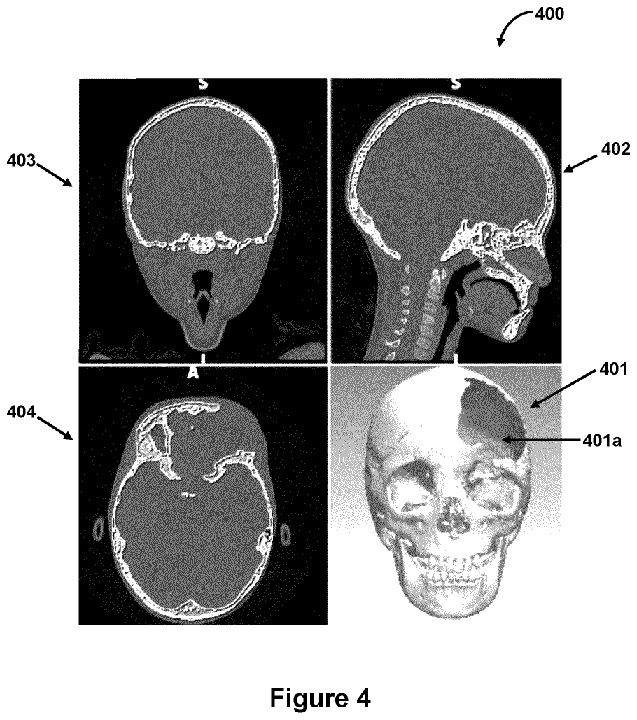

In an embodiment, the system 100 is configured to integrate different types of software applications and algorithms (e.g., see FIG. 4) to generate a manufacturing workflow specific to the user-specific device. The manufacturing workflow may include a sequence of tasks to be performed by one or more entities at a given point in time. The tasks may be determined based on the user-specific information associated with a requested user-specific device. Thus, the system 100 enables automatic determination of specific workflows suitable for a requested user-specific device. For example, generating the workflow includes generating appropriate questions or information via the dynamic input generation subsystem 112, generating a device design based on the received user-specific information via the model generation subsystem 114, receiving a feedback and approval via the feedback subsystem 118, and/or generating manufacturing steps for manufacturing the user-specific devices via a project generation subsystem 116.

Processor 102 may include dynamic input generation subsystem 112, model generation subsystem 114, project generation subsystem 116, feedback subsystem 118, graphical user interface subsystem 120, or other components. Each client device 104 may include any type of mobile terminal, fixed terminal, or other device. By way of example, client device 104 may include a desktop computer, a notebook computer, a tablet computer, a smartphone, a wearable device, or other client device. Users may, for instance, utilize one or more client devices 104 to interact with one another, one or more servers, or other components of system 100. It should be noted that, while one or more operations are described herein as being performed by particular components of processor 102, those operations may, in some embodiments, be performed by other components of processor 102 or other components of system 100. As an example, while one or more operations are described herein as being performed by components of processor 102, those operations may, in some embodiments, be performed by components of client device 104.

The client devices 104a-104n may be configured to display a graphical user interface that displays, receives, and/or allows manipulation of user-specific information. The graphical user interface manages the interaction between a computer system and different entities through graphical elements such as windows on a display. The graphical user interface enables efficient exchange of different type of information between different entities. Based on the information exchanged via the graphical user interface, the system 100 determines a sequence of tasks to be performed by different entities or the computer system in order to control the designing and manufacturing of user-specific devices.

In an embodiment, the information exchanged via the graphical user interface may be related to a structure and functions of the user-specific devices, designing task of the user-specific devices, feedback task related to the designed devices, and/or manufacturing tasks of the user-specific device. These tasks may be performed by different entities at different point in time. As such, an integration of various tasks into a manufacturing workflow is beneficial so that each entity can view, analyze, and provide timely feedback before manufacturing the actual user-specific device. In an embodiment, the processor 102 includes exemplary subsystems that enables seamless integration of these process so that end-to-end manufacturing process of user-specific devices may be controlled by the requesting entity and/or the second entity.

In some embodiments, the dynamic input generation subsystem 112 may be configured to generate a data input request to receive user-specific inputs from the requesting entity E1. In an embodiment, the generated data input request conforms with requirements of a subject entity. In an embodiment, the data input request may include user-specific questions that may be displayed via the graphical user interface subsystem 120 to the requesting entity E1 and/or other users having secured access to the system 100. In an embodiment, the dynamic input generation subsystem 112 may request a high-level information (e.g., an intended use, a condition of the user, a procedure in which the device may be used, a desired outcome, etc.) from the requesting entity E1 to determine a type or category of devices desired by the requesting entity E1 that may potentially improve issues faced by a user (e.g., the requesting entity or a subject entity). Once the type or category of devices is determined, the dynamic input generation subsystem 112 may generate additional questions regarding user-specific data to modify or design one or more devices for the requesting entity E1. As an example, the dynamic input generation subsystem 112 may securely communicate with a device database 132 to extract device information related to one or more devices that may help with the user-specific problems and request additional information from the requesting entity E1 based on the extracted device information.

In some embodiments, the system 100 may receive the user-specific inputs via a graphical user interface that is dynamically configured to include input fields to receive user-specific data (e.g., structural or procedural constraints). In an embodiment, the system 100 may dynamically update one or more input fields based on the user-specific data entered by the requesting entity so that appropriate information related to one or more user-specific devices may be collected and used during the designing and/or manufacturing process. In some embodiments, upon receiving the user-specific inputs, the system 100 may transmit the user-specific inputs to a second entity for designing of the one or more devices.

In some embodiment, system 100 may be configured to manufacture a patient-specific medical device. A medical device may be any device intended to be used for medical purposes. Medical devices benefit patients by helping health care providers (e.g., a surgeon) diagnose and treat patients, helping patients overcome a medical condition such as sickness or disease, and improving the patient's quality of life. Medical devices are associated with several constraints when using a device for medical purposes including structural, functional, regulatory compliance associated with a geographical location e.g., country, state, or other constraints related to treatment or surgery procedures. As an example, the medical device may be an anatomical model of the body part of a subject entity (e.g., a patient), a surgical guide to be used for a surgery by a requesting entity (e.g., a surgeon); or an implant for the body part of the subject entity. As additional example, one or more user-specific device may be a devices used to improve a medical condition of a patient such as a patient specific face mask. Patients may have unique characteristics such as body part structures, medical issues, etc. which constraints the design and manufacturing of the patient-specific device.

In an embodiment, the device characteristics may be at least one of: a particular material based on the intended use of the device; a customized geometry to fit the structure of the body part during a particular treatment cycle; and a sub-component or a particular area of interest of the structure of the body part. In an embodiment, the particular material has one or more material property comprising: biocompatibility, flexibility, durability, transparency, utility, life-like appearance, or a combination of properties.

In an embodiment, the intended use of the device comprises at least one of: a pre-surgical planning by a requesting entity; an intra-surgical device used by the requesting entity; visual communications (e.g., with a group or a patient); surgical simulation prior to the intended procedure; a post-surgical procedure to be performed by the requesting entity or the subject entity; and an implantation in the body part of the subject entity.

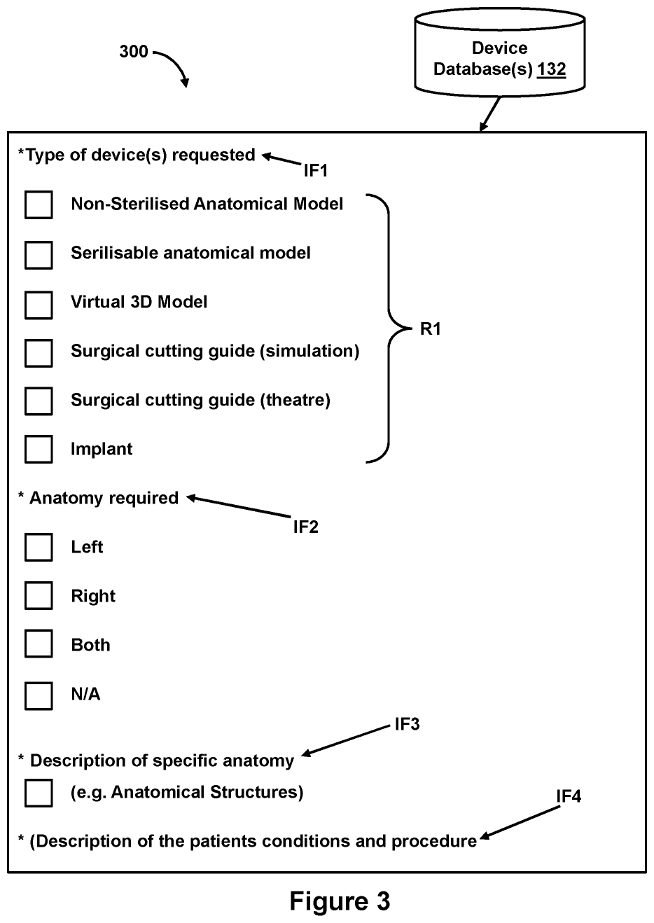

FIG. 3 illustrates an exemplary output of the dynamic input generation subsystem 120 displayed on a graphical user interface 300 for determining a patient-specific device (an example of a user-specific device) and the manufacturing process therefor. The dynamic input generation subsystem 120 may initially receive a first set of information such as intended use, desired outcome, type of application, or other high-level information related to a user (e.g., a patient) or the patient specific device. In an embodiment, the first set of information may be a volumetric scan of a patient. For example, FIG. 4 illustrates the volumetric scan 400 of a head of a patient. The volumetric scan includes different views such as an isometric view 401, a left side view 402, a side view 403, and a top view 404. In an embodiment, the requesting entity provides the intended use of the patient-specific device. For example, the intended use may be associated with a broken portion 401a of the skull. In an embodiment, the system 100 may perform an image analysis on the volumetric scan of the head to determine a geometry of the broken portion and compare it with devices in the device database 132 that may provide a solution to fix the broken portion 401a. In an embodiment, one or more solutions may be available based on historic information.

Referring back to FIG. 3, based on the first set of information, the dynamic input generation subsystem 120 may generate a second set of information to receive additional information related to the patient-specific device. The second set of information may be related to designing and/or manufacturing flow of the patient-specific device. In an embodiment, the second set of information is generated by comparing the first set of information with characteristics of a list of devices in the device database 132. The second set of information may be further displayed on the graphical user interface 300 so that the requesting entity can provide more specific information that may affect the designing and/or manufacturing flow of the patient-specific device. Thus, the graphical user interface 300 is dynamically updated to proactively collect patient-specific and/or patient-specific device information so that interaction between the first and second entity may be minimized while enabling more accurate information exchange.

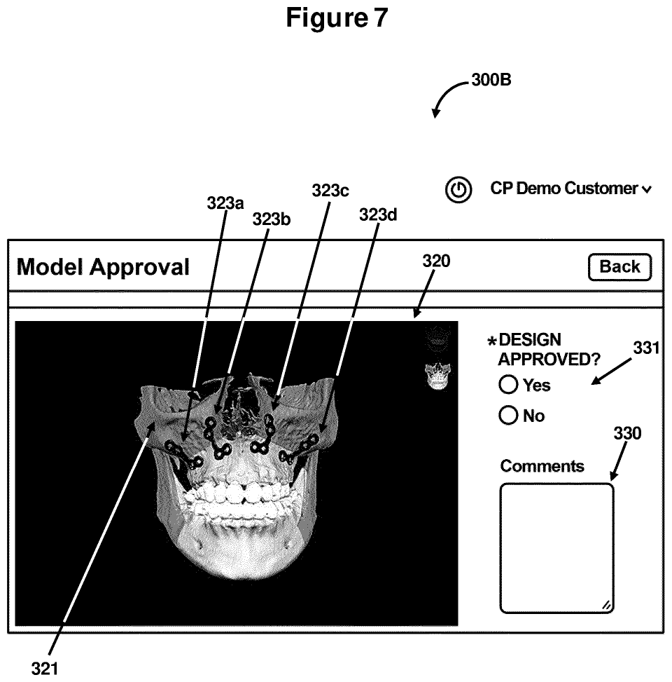

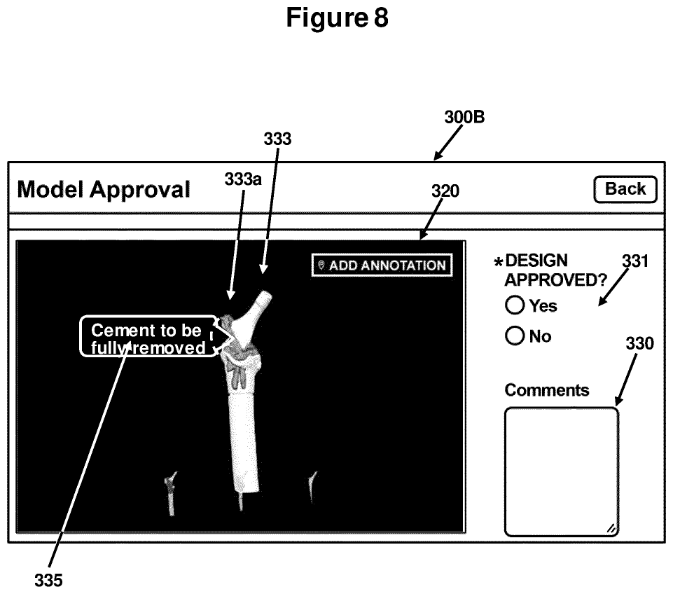

In some embodiments, the graphical user interface subsystem 120 may include a plurality of separate interfaces associated with computing device 104a, and/or other subsystems of the system 100, for example. In some embodiments, the graphical user interface subsystem 120 includes at least one interface that is provided integrally with a computing device 104a or 104b. As an example, the graphical user interface 300 comprises a first interface or screen having one or more input fields (e.g., IF1, IF2, IF3, IF4) configured to receive entry and/or selection of the patient-specific device information from the requesting entity, a second interface or screen having one or more display fields configured to display a virtual representation of the patient-specific devices (e.g., see FIGS. 7 and 8) to the requesting entity and/or other users, a third interface or screen having one or more feedback fields configured to receive feedback on the virtual representation, a fourth interface or screen having one or more approval fields configured to receive an approval from the requesting entity. In an embodiment, one or more portions of different interfaces may be integrated into a single interface or screen to effectively display information in a compact manner.

In an embodiment, the graphical user interface 300 is displayed on a computing device 104a for the requesting entity, but not on the computing device 104b configured for the second entity. The graphical user interface 300 may be displayed on the computing device while the requesting entity is in at home, at a hospital, and/or at other location or times. The graphical user interface 300 may be configured to provide an interface between the computing device 104a and the requesting entity through which the requesting entity may provide information to and receive information from the second entity. This enables patient-specific information, design information, manufacturing progress, desired results, feedback, and/or instructions and any other communicable items, collectively referred to as “information,” to be communicated between the requesting entity and the second entity or the system 100.

In an embodiment, the graphical user interface 300 is a client facing portal of the system 100 that is configured as a secured log-in based web-portal to allow the requesting entity or the second entity to securely login, provide patient-specific information and access patient-specific device design/manufacturing results. The graphical user interface 300 may also be configured to approve one or more patient-specific device that are being worked on for the requesting entity. The graphical user interface 300 may also be configured to receive feedback at an end of the design or the manufacturing process, thereby providing a closed loop workflow to enable end-to-end control of the patient-specific device manufacturing process.

In some embodiments, the graphical user interface 300 is dynamically changed from a simple form to a more complex form to receive/display use-specific details. For example, a simple questionnaire about a request may be displayed to receive high-level information from the requesting entity. The high-level information may be, for example, a type of application, structure of a body part, an intended use of the device, and intended outcome upon use of the device. Based on this information, a logic may be executed by the system 100 to determine a list of devices or category of devices that correspond to the rudimentary information. For example, the list of devices may be selected from the device database 132 by matching the device characteristics, and the intended use of the device.

Accordingly, the dynamic input generation subsystem 112 and the graphical user interface subsystem 120 may communicate with each other to cause the graphical user interface 300 to dynamically change and populate new input fields showing the list of devices. For example, as shown in FIG. 3, the graphical user interface 300 illustrates options R1 showing a list of devices that may be used to address the issues of the user. In an embodiment, if the requesting entity requests a particular model or a particular product in the list of options R1, the graphical user interface 300 may be further changed to include additional fields (e.g., IF2, IF3, and/or IF4) or questions to correspond to the different products or model that is requested.

In some embodiments, based on the selected device (e.g., an implant, virtual 3D model, sterilizable anatomical model), the second entity may also request additional information. For example, when the requesting entity selects implant option from the options R1, an input field IF4 may be included on the graphical user interface to request a corresponding tool that may be used to install or implant the device. For example, the tools such as a screw-driver, a drill, etc. may be used in cooperation with a patient-specific device. As such, information about the tool may also be desired by the second entity to determine a structure of the device. In an embodiment, the graphical user interface may be updated to request additional information of the tools that may be cooperatively used with the patient-specific device. In this way, advantageously, the graphical user interface reduces the back and forth between different entities to implement design changes that may be required, and reduce the changes of unusable or faulty manufactured patient-specific device. Thus, the design and manufacturing process can be improved via the dynamically updating the graphical user interface.

In some embodiment, based on a geographical location, certain regulatory documents may be required for a particular device (e.g., implants). The system 100 may be configured to generate such documents and display on the graphical user interface 300. Thus, the graphical user interface 300 may serve as a single client portal where information (e.g., including documents, images, text, virtual models, etc.) related to the user-specific devices may be received, displayed, accessed, and/or manipulated.

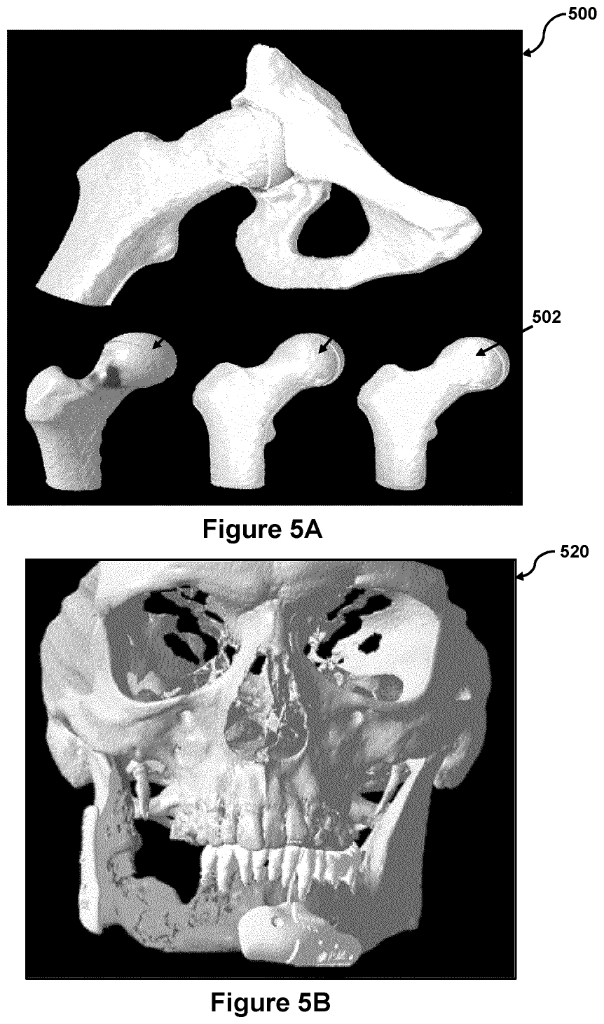

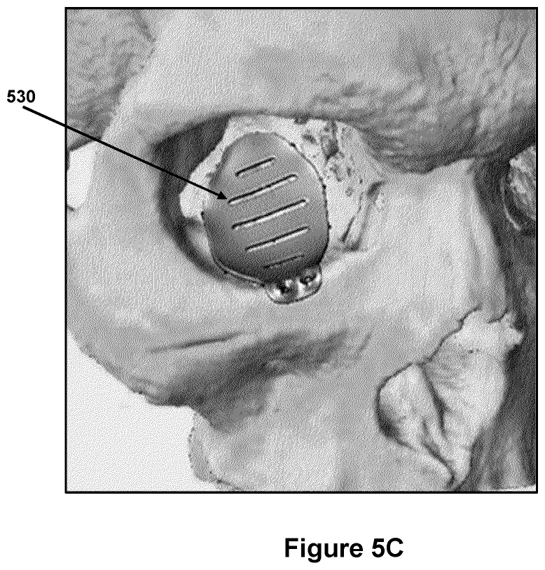

FIGS. 5A-5C illustrates exemplary patient-specific devices that may be selected based on the desired outcome, the intended use, and/or the type of application of the devices. FIG. 5A is an interactive digital model 500 of a hip portion of a patient. In an embodiment, the system 100 may recommend a first user-specific device in a form of a digital model 500 mimicking the patient-specific anatomy of the hip portion. In an embodiment, a plurality of orientation of a portion of the hip may also be provided. For example, different orientations 502, 504, and 506 of a joint portion is provided. In an embodiment, the digital model 500 may be used in conjunction with the operating tools (that may be used during the surgery) to simulate the surgery procedure. In an embodiment, the one or more patient-specific devices may provide a realistic understanding of access points for operating tools (not illustrated), orientations of joints of the hip, etc. so that the surgery can be performed efficiently and effectively to obtain a desired hip replacement effect. In an embodiment, if the intended use of the patient specific device is to perform a hip replacement surgery, the system 100 may determine that a second user-specific device should be manufactured from a bone-like material so that a surgeon may prepare for the surgery on a dummy device prior to performing the actual surgery. As such, the surgeon may practice the procedure of the surgery to improve the outcome of the actual surgery.

FIG. 5B illustrates an interactive digital model 520 of a skull. For example, the interactive digital model of skull is a surgical cutting guide or drilling guide to be used for the surgery. In an embodiment, the skull may be used in a similar manner as the hip portion FIG. 5A discussed above. In an embodiment, the interactive digital model of the skull may be used to analyze access points of a brain surgery.

FIG. 5C illustrates an interactive digital model 530 of an orbital floor implant. The orbital floor implant will have a customized geometry to fit a specific patient's anatomy at a particular stage of their treatment cycle. This type of implant is typically used to repair damage which would cause muscles or other orbital contents to enter the maxillary antrum. For procedures using implants such as the orbital floor implant, extreme care must be taken on placing and fixing the implant to allow long term implantation. An exact fit of the implant to conform to the shape of the orbital portion is desired and therefore material selection, manufacturing technology and appropriate design are essential. The system 100 may be configured to select not only the appropriate material (e.g., titanium) based on the type of implant, but also control third party manufacturing workflows (e.g., a sequence of tasks the third party should complete within a time period), suggest an accompanying model (e.g., a model of an orbital area surrounding a subject's eye made from a bone like material, or a digital model) and also a cutting guide to assist with more accurate placement/fixing. In an embodiment, the system 100 may also ensure that relevant regulatory documentations related to orbital implants are produced. As another example, for atrial septal defect cardiac surgery only an anatomical model may be available.

The information received from the graphical user interface subsystem 120 (e.g., via the graphical user interface 300) is further transmitted to another subsystem such as the model generation subsystem 114, and the project generation subsystem 116. In some embodiment, the graphical user interface subsystem 120 may communicate with a feedback subsystem 118 and modify the graphical user interface (e.g., to display a feedback or approval interface shown in FIGS. 7 and 8). In an embodiment, these subsystems may each be cloud-based software application.

Referring back to FIG. 2, in some embodiments, the model generation subsystem 114 is configured to generate a digital interactive model by the second entity based on the user-specific information provided by the graphical user interface subsystem 120. The interactive digital model may be a three-dimensional (3D) digital model of a device, a two-dimensional image (e.g., top view, side view, cross-section view or other views of 3D model), a 3D digital structure of the body part, or other digital models that may be used by the requesting entity as a guide to better understand and resolve issues of the subject entity. The interactive digital model is generated based on the user-specific inputs provided by the requesting entity. For example, the user input includes type of surgery, intended use of the device, desired output of the surgery, a procedure of a surgery, an assembly process, etc.

In an embodiment, the model generation subsystem 114 is securely accessed by the second entity to design one or more interactive digital models of one or more user-specific devices using the user-specific inputs received via the graphical user interface subsystem 120. In an embodiment, the model generation subsystem 114 may be configured to convert the interactive digital model into a format that is importable by the graphical user interface subsystem 120 and displayed in a portion of the graphical user interface (e.g., see FIGS. 7 and 8).

In an embodiment, the model generation subsystem 114 generates the design of the requested device based on the user-specific information collected via the dynamic input generation subsystem 112. In an embodiment, the generating of the device design involves determining, by the designing entity, a location and an orientation within the subject entity's environment, a geometry of the device based on the user-specific information provided by the requesting entity, a material of the user-specific device compatible with a structure of the body part or the intended use of the device, or other structural characteristics of the user-specific device. In an embodiment, the information provided by the requesting entity may include scans or schematics of the subject entity from which the designing entity may derive the geometry (e.g., size, shape, etc.) of the user-specific device.

In an embodiment, the model generation subsystem 114 may also determine the manufacturing steps of the one or more user-specific devices. The manufacturing process may include one or more manufacturing tools configured to manufacture geometric shape, density, surface finish and/or other properties of a device. For example, the one or more manufacturing tools may be a three-dimensional (3D) printer configured to receive the design of the device and print the device using suitable material based on the intended use of the device; a controlled manufacturing environment, where one or more parameters of the manufacturing tool are controlled to manufacture the device; a post-processing equipment configured for cleaning, curing, or finishing the device, or other apparatuses that may be based in manufacturing process. In an embodiment, the controlled manufacturing environment is maintained with particulate levels and microbial counts below a safe threshold ensuring suitable cleanliness of the user specific device.

In some embodiments, the system 100 includes the project generation subsystem 116 that is configured to generate a project workflow based on information derived from the user-specific inputs received via the graphical user interface subsystem 120. In an embodiment, the workflow comprises various stages (e.g., designing, feedback, approval, and actual manufacturing steps) of the user-specific device manufacturing process configured to manufacturing of the user-specific device.

In an embodiment, the project generation subsystem 116 may be configured to enable decision making related to a combination of factors including, but not limited to, workflow decisions and regulatory compliance related decisions that are specific to each requested user-specific device. In an embodiment, the project generation subsystem 116 creates a set of template projects that correspond to each device family. Further, project generation subsystem 116 employs an algorithmic approach to optimize the workflow based on the user-specific inputs or feedback, while allowing the requesting entity to modify the workflow based on any particular requirements during feedback stage. In this manner, the system 100 provides a technology platform configured to control the data exchange as well as workflow decisions based on the user-specific requirements.

As an example, the project generation subsystem 116 employs an algorithm configured to determine an initial workflow for a general category of device request by the requesting entity, and further optimize the initial workflow based on user-specific data to manufacture a user-specific device. In an embodiment, the term workflow refers to a set of steps to be taken from an initiation step to manufacturing of the user-specific device. For example, the steps include, but not limited to, designing a device based on user-specific data, receiving feedback (e.g., via the graphical user interface modified by the feedback subsystem 118) on the device design, modifying the design based on the feedback, identifying resources to incorporate the design changes, and updating manufacturing steps based on the changes to the design.

As an example, the algorithm, at first, suggests a suitable solution (e.g., type of device and characteristics) to the requesting entity and displays the solutions via the graphical user interface 300, and simultaneously modifies, based on user-inputs, a dynamic electronic form (e.g., generated by the dynamic input generation subsystem 112) to receive user-specific data corresponding to the solution. As discussed, the dynamic electronic form refers to dynamically updatable form based on user inputs so that appropriate user-specific data may be collected to optimize the workflow solution. In an embodiment, the dynamic electronic form may be a questionnaire configured to receive general information about a device from the requesting entity. Further, based on the inputs from the requesting entity, the questionnaire can be updated to populate questions or input fields for receiving user-specific data based on the user-specific data. In an embodiment, the dynamic electronic form may be displayed on a graphical user interface.

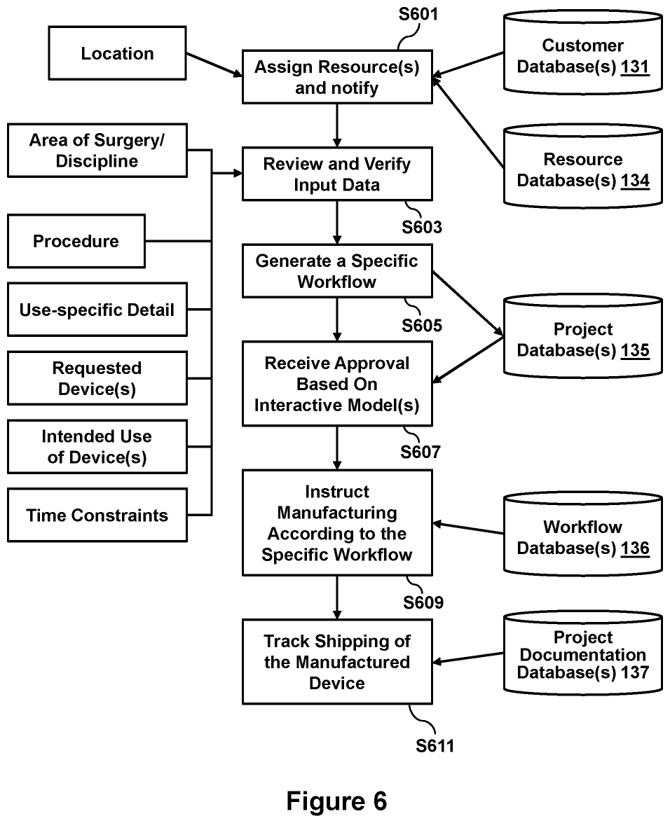

As mentioned earlier, the dynamic electronic form (e.g., displayed on GUI 300 in FIG. 3) may present a first set of questions to the requesting entity. Based on the response to the first set of questions, a first workflow may be extracted from a workflow database (e.g., database 136 in FIG. 6) configured to store a sample manufacturing process, for a particular category of devices, a particular procedure to be performed using the device, etc. As the sample process is generic for the particular category of devices, further optimization is needed to incorporate user-specific data into the workflow. Such user-specific data may significantly impact the design as well as manufacturing process of the sample workflow related to the category of the devices.

In an embodiment, once the optimal solution has been determined, the workflow for the design and manufacture of that device may be displayed and monitored to ensure timely manufacture of the device. In an exemplary optimized scenario, a requesting entity may submit volumetric patient scan data (e.g., see FIG. 4) and an operating procedure associated with the device. Based on these inputs, the project generation subsystem 116 determines a suggested solution workflow that are patient-specific and support the desired outcome along with pricing and lead times between initiation and completion of manufacturing of the device.

Due to the highly variable workload associated with patient-specific device design and manufacture, the project generation subsystem 116 may implement a prediction model configured to determine resources, timelines and cost based on input data related to the user-specific device received via the graphical user interface. In some embodiments, the model may receive user-specific data, feedback data, or additional in-project changes to determine resources, timelines and costs associated with the changes. The project generation subsystem 116 may support both modification of an initial workflow as well as track actual time spend on deliverables, and variations to continually improve the accuracy of the model predictions.

In some embodiments, the project generation subsystem 116 allocates one or more resources for designing and/or manufacturing of the device based on educational background, skill sets, timely availability of resources, capability of the one or more resources that are compatible with the user-specific information, and/or the feedback received from the requesting entity. For example, a customer and/or an engineer resource details may be retrieved from a customer database 131 and the resource database 132, respectively. In an embodiment, the resource database 132 comprises information about potential designing and manufacturing entities, (e.g., the engineers of medical devices, and 3D printers together with the engineer's specializations). In an embodiment, a dynamic view of resource availability and the manufacturing tools may be made available by the system 100. In some embodiments, allocating the one or more resources involves predicting an amount of time and a number of personnel required for designing and/or manufacturing of the device. In an embodiment, the allocating may also predict a total cost of the design and manufacture of the user-specific device. Based on the time and cost, a determination can be made whether additional resources (e.g., third party manufacturers, vendors, consultants, etc.) than the designing and manufacturing entity be used in the manufacturing workflow.

In some embodiments, allocating the one or more resources involves distributing of the interactive digital model or the updated interactive digital model associated with the device to one or more personnel across distributed sites; and/or distributing of the manufacturing of the device to one or more distributed sites. In some embodiments, allocating the one or more resources involves assigning, based on a geographical location of the requesting entity, the one or more resources of the second entity that are within a desired region of the requesting entity. Such distribution of the resources based on geographical proximity to the requesting entity can be advantageous for timely delivery even when several user-specific changes may be requested by the requesting entity late in the manufacturing process. In this way, based on user-specific inputs derived from response received from the requesting entity via the graphical user interface (e.g., 300), resource allocation process may be incorporated into determining manufacturing workflows. This enables making changes to the workflows in real-time without significantly affecting a delivery time of the user-specific devices.

In an embodiment, the project creating subsystem 116 involves generating and assigning of a unique identifier to the user-specific device. Based on the unique identifier, changes to the interactive digital model associated with the user-specific device made by the requesting entity, and/or the second entity may be tracked. In some embodiments, based on the unique identifier, a progress of manufacturing of the user-specific device may be tracked. For example, one or more tasks within the workflow may be tracked and monitored to advantageously track whether appropriate changes to the design and manufacturing are incorporated. Responsive to changes not being incorporated, appropriate control measures may be taken by the manufacturing entity or the design entity to incorporate design or manufacturing changes without affecting a time period of device delivery. In some embodiments, based on the unique identifier, data (e.g., a design file associated with the interactive digital model, changes to the design, feedback, etc.) associated with the user-specific device may be stored and retrieved from a device database. In an embodiment, based on the unique identifier, the manufactured device can be identified when received by the requesting entity.

In some embodiment, the project generation subsystem 116 receives a geographical location of the requesting entity as a part of the information received via the graphical user interface. Based on the geographical location, the relevant jurisdiction, regulatory specifications and/or quality assurance constraints related to the user-specific device for the that jurisdiction may be determined. Further, the manufacturing workflow for the designing and manufacturing entities may be generated or a previously selected manufacturing workflow may be updated to comply with the requirements of the jurisdiction. In an embodiment, regulatory and quality assurance documentations may be generated to meet the regulatory specifications of the geographical location. In some embodiments, the updated interactive digital model and/or the updated manufacturing workflow associated with the user-specific device may be stored in the device database and transmitted to manufacturing.

FIG. 6 illustrates an exemplary flow chart of an algorithm implemented by the project creating subsystem 116 to generate a workflow for patient-specific medical devices. In FIG. 6, the project generation subsystem 116, at step S601 involves resource assignments configured to assign resources for designing and/or manufacturing the patient-specific medical devices based on the patient-specific inputs. At step S601, the project generating subsystem 116 may extract information from the customer database 131 and the resource database 132 based on patient specific medical devices.

Step S603 involves receiving and verifying input data (e.g., volumetric scan) so that a corresponding patient-specific medical devices is compatible with the patient. For example, the patient-specific data may include an area of surgery or discipline (e.g., arthroscopy, hip replacement, cataract, dental restoration, endocrine, head surgery, knee surgery, etc.), a surgery or treatment procedure, user-specific details (e.g., structure of body parts, medical conditions, etc.), requested medical devices, intended use of device(s), time constraints, or other user-specific inputs that may affect (e.g., cause structural changes) the designing and/or manufacturing steps.

Step S605 involves generating a set of manufacturing steps by redefining or updating a workflow template associated with a type of medical device to incorporate patient-specific requirements. The workflow may be stored in a project database 135 to extract project information, monitor progress of a project (e.g., manufacturing of a patient-specific medical device), track changes to the design or manufacturing, or other project activities. For example, when requesting entity requests for an anatomical model device used for medical diagnostics and planning, the system 100 will automatically assign an anatomical model workflow determined by sterilization requirements of the device. A procedure specified by the requesting entity may determine manufacturing equipment and material to be used, for example wood-based materials can simulate realistic bone properties for orthopedic surgery planning or testing. All of these steps will be incorporated into the project workflow and in turn each of the workflow tasks can completed by a project manager allowing the progress and success of the project to be monitored.

Step S607 involves receiving approval from the requesting entity based on an interactive digital model so that appropriate changes can be made to the design and/or manufacturing steps of the patient-specific medical devices. For example, the approval may be received via the graphical user interface 300 and stored in the project database 135 so that the second entity may access the project information to view the approval and proceed with manufacturing steps.

Step S609 involves sending instructions and controlling the manufacturing process of a manufacturer of the patient-specific medical devices according to the approved interactive digital model. Step S611 involves track shipping of the manufactured patient-specific medical devices to ensure timely delivery to the requesting entity so that appropriate medical procedure may be performed within reasonable time. At step S611, project documentation database 137 may be accessed to generate medical device-specific and location-specific regulatory and compliance documentation to be included in the delivery package of the medical devices.

Referring back to FIG. 2, the feedback subsystem 118 may be configured to create a proactive environment to adjust the device design and/or the user-specific manufacturing process. In some embodiments, the feedback subsystem 118 is configured to receive, via the graphical user interface subsystem 120, feedback from the requesting entity on the interactive digital model. In some embodiments, the feedback includes comments, highlights on a portion of the interactive digital model, potential errors in structure of the user-specific device, and/or desired modifications associated with the interactive digital model.

Since user-specific devices (e.g., patient-specific medical implants) may have structure and function specific to a user (e.g., a patient), feedback (e.g., from the surgeon or the patient) is gathered on a case-by-case basis. The feedback subsystem 118 may prompt a requesting entity to record their feedback dynamically based on the nature of the user-specific device, timelines related to the manufacturing and delivery, or other feedback. The feedback subsystem 118 advantageously creates a proactive environment for post-market surveillance that is sensitive to the custom nature of the device being delivered, rather than assuming mass production of a particular item and attempting to gather identical feedback from a much larger user population.

In some embodiment, the feedback subsystem 118 communicates with the graphical user interface subsystem 120 to enable the user-device visualization and approval. In an embodiment, the feedback subsystem 118 modifies the graphical user interface (e.g., 300) to receive feedback the requesting entity (e.g., a surgeon) on the interactive digital model before manufacturing a patient-specific medical device. Such visualization and approval may be desired from both regulatory and quality perspectives, as well as to improve awareness and confidence that the proposed patient-specific device will meet patient-specific requirements.

In an embodiment, the feedback subsystem 118 may generate a relevant anatomy compatible with the user-specific device (e.g., an implant) and cause the graphical user interface to display the interactive digital model as a digital model (e.g., 2D image, 3D image) in a 3D viewer within a portion the graphical user interface. The requesting entity may review, analyze by orienting and reorienting the user-specific device, analyze the device with respect to the anatomy of the body part, remove or omit certain features of the medical device or body parts for better understanding and analysis of the anatomy or fit of the medical devices, etc. Based on the review and analysis, the requesting entity may provide feedback in terms of annotations, highlights, or comments on the interactive digital model. In an embodiment, the feedback subsystem 118 may receive to feedback and other manipulation information from the graphical user interface.

In some embodiment, the second entity may tag or highlight certain portions of the user-specific device to bring it to the attention of the requesting entity. Such tagging or highlighting may be related to acquiring further user-specific structure, analyzing the structure of the device, or other purposes affecting the structure or manufacturing of the user-specific device. Upon reviewed the interactive digital model or the updated interactive digital model, the requesting entity can indicate approval of the update interactive digital model via the graphical user interface. The feedback subsystem 118 can then forward the feedback and/or approval to the second entity for further updating and manufacturing the user-specific device.

FIGS. 7 and 8 illustrates exemplary patient specific medical devices and feedback mechanism implemented via a graphical user interface, according to an embodiment. FIG. 7 illustrates an interactive digital model including a jaw anatomy 321 and dental implants 323a-323d superimposed on the jaw anatomy 321, which is specific to a patient. In an embodiment, the jaw anatomy 321 is specific to the patient, and the structure of implants is configured to cooperate with the jaw anatomy 321. For example, the structure of the implants 323a-323d is designed to fit in an orientation with respect to the jaw of the patient. For another patient, a different implant and orientation may be needed.

In this example, the interactive digital model 321 is displayed as a digital image on a graphical user interface screen 300A. A healthcare professional such as a dentist may manipulate the interactive digital model 321 and provide feedback on the implants 323a-323d. The interactive digital model 321 may be rotated or reoriented to view the implant 323a-323d from different viewpoints to ensure appropriate fit with respect to the jaw structure. The dentist may analyze the implants 323a-323d to ensure the implants do not interfere with other parts of the jaw. For example, the feedback may include point to a specific portion of an implant (e.g., 323a) and commenting on desired changes in the structure of the implant. As an example, the changes may be increase or decrease a size of a portion of the implant to avoid interference with parts (e.g., sinus) surrounding the jaw, reorienting one or more of the implants 323a-323d to provide better access points for installing the implants. Based on the feedback, the second entity can make appropriate structural changes and update the interactive digital model 321 and the implants 323a-323d. Final approval on the updated interactive digital model and implants may be obtained via an approval field 331 or comment box 330. Once approved, the implants 323a-323d, the jaw anatomy 321, or both may be manufactured.

FIG. 8 illustrates another exemplary interactive digital model 333 of a knee portion of a patient. In some embodiments, the knee interactive digital model 333 is also patient-specific since different patients may have different knee structure due to wear and tear, dislocation due to injury, or other patient-specific issues. In an embodiment, the requesting entity may be a knee surgeon who may be interested in analyzing the knee structure and issues with knee replacement procedures before performing the knee surgery. As such, the knee surgeon may desire a knee model having bone like material configured to have the patient-specific knee structure. Once, an initial interactive digital model is generated, the feedback subsystem 118 may modify the graphical user interface (e.g., 300) to display the interactive digital model 333 on a portion of the graphical user interface 300B configured to include a 3D interactive digital model portion 320, approval field 331, and comment box 330. Similar to feedback discussed above, the knee surgeon may provide feedback on the interactive digital model 333. For example, the knee surgeon may highlight portions 333a of the knee model 333, remove one or more portion of the knee model to analyze the structure of a portion of the knee structure, or other manipulations, provide annotations and comments 335 at specific locations of the knee, etc. As discussed above, the second entity may update the interactive digital model 333 based on the feedback. Once the updated model is approved by the knee surgeon, the interactive digital model 333 may be manufactured.

### Example Flowchart(s)

The example flowchart(s) described herein of processing operations of methods that enable the various features and functionality of the system as described in detail above. The processing operations of each method presented below are intended to be illustrative and non-limiting. In some embodiments, for example, the methods may be accomplished with one or more additional operations not described, and/or without one or more of the operations discussed. Additionally, the order in which the processing operations of the methods are illustrated (and described below) is not intended to be limiting.

In some embodiments, the methods may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of the methods in response to instructions stored electronically on an electronic storage medium. The processing devices may include one or more devices configured through hardware, firmware, and/or software to be specifically designed for execution of one or more of the operations of the methods.

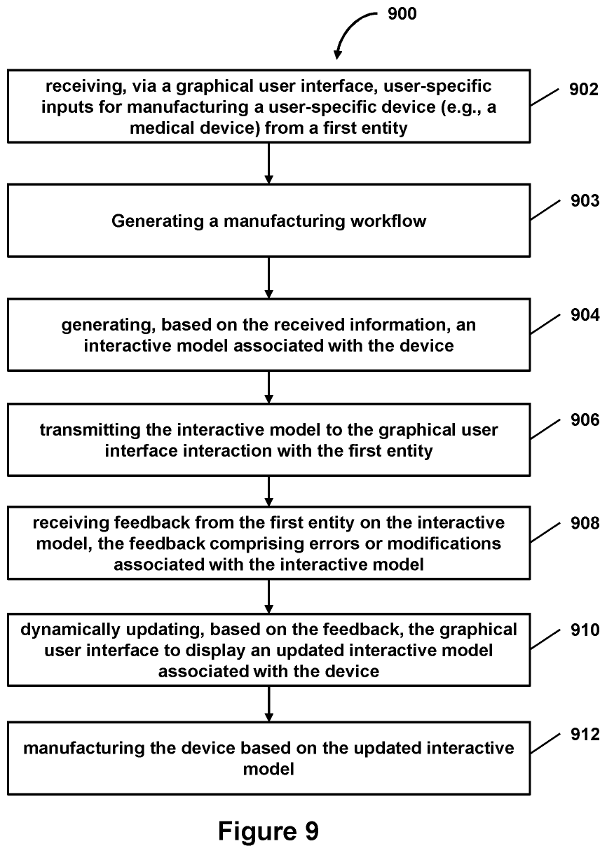

FIG. 9 is an exemplary flowchart of a method 900 for controlling manufacturing process of a user-specific device (e.g., patient specific medical device), according to an embodiment.

At operation 902, user-specific inputs for manufacturing a user-specific device (e.g., a medical device) may be received from a requesting entity via a graphical user interface (e.g., interface 300 in FIG. 3), where the inputs conform to a subject entity's requirements. In an embodiment, the graphical user interface is configured to include patient-specific information related to a medical procedure (e.g., dental implant procedure, knee surgery, hip replacement) to be performed by a healthcare provider (e.g., a surgeon, clinician, etc.). In an embodiment, the patient-specific information comprises a type of application (e.g., a type of surgery), environment of the subject entity (e.g., anatomy of a body part) in which the user-specific device may be deployed, an intended use of the medical device, and intended outcome upon use of the medical device. In an embodiment, the operation 902 includes determining a list of user-specific devices (e.g., medical devices) and device characteristics based on the user-specific information (e.g., the patient-specific information) related to the medical procedure. The list of devices may be displayed on the graphical user interface for the requesting entity to make a selection. In an embodiment, the requesting entity may select one or more devices. Further, responsive to selection of the one or more devices from the list of devices (e.g., medical devices), additional user-specific (e.g., patient-specific information) to be requested from the requesting entity may be determined.

As an example, an initial list of devices may be a patient-specific medical device selected from a list of: anatomical models of the body parts; a surgical guide to be used for a surgery; and an implant used to surgically replace a bone, tissue, organ, or other implants. The requesting entity may select from the list of devices, a device to be configured as a user-specific device to be manufactured. In an embodiment, the second set of information may be generated by determining, based on the selected one or more devices and the received information, suggested complementary or alternative device to the user-specific device selected by the requesting entity. The complementary or alternative device may be, for example, a surgical guide, an anatomical model made of bone or flesh like material complementing the user-specific device, a simulation model, or other devices.

The device characteristics may include, but not limited to, a particular material based on the intended use of the patient-specific medical device; a customized geometry to fit the structure of the body part during a particular part of the medical procedure (e.g., dental implant procedure, knee surgery, hip replacement); and a sub-component or a particular area of interest of the structure of the body part. In an embodiment, the particular material has one or more material properties including biocompatibility, flexibility, durability, life-like appearance, or a combination of properties. In an embodiment, the intended use of the device (e.g., the medical device) may be at least one of: a pre-surgical planning; an intra-surgical; visual communications with a group or a patient; a surgical simulation of a procedure to be performed on the body part; a post-surgical procedure; and an implantation in the body part.

In an embodiment, receiving the patent-specific information involves receiving volumetric scan data of a patient and steps of the medical procedure to be performed in the surgery. In an embodiment, receiving the list of medical devices involves selecting, based on the information of the medical device from a device database, the list of medical devices matching the medical device characteristics, and the intended use of the medical device in the medical procedure associated with the patient.

In some embodiment, operation 902 may be performed by a subsystem that is the same as or similar to the dynamic input generation subsystem 112 and the graphical user interface subsystem 120, in accordance with one or more embodiments.

At operation 903, a manufacturing workflow may be generated based on the user-specific inputs received via the graphical user interface. For example, the project generation subsystem 116 may generate the manufacturing workflow based on a workflow in a workflow database 136 (in FIG. 6) using the one or more selected devices as a guide. The workflow may include a plurality of taks to be performed by different entities, or the computer system (e.g., processor 102) to design and manufacture the user-specific device within a desired time. In an embodiment, the workflow includes a timeline for each task, triggering events along the timelines, etc. The triggering events may be, for example, a request triggered by the system 100 or one or more entities during a manufacturing process of the user-specific device. For example, a request may be initiated by the system or a designing entity for requesting additional information from the requesting entity at a start of the project. As another example, a request may be triggered by the designing entity for requesting feedback from the requesting entity after completing a design. As yet another example, a notification may be triggered by the system 100 when the requesting entity approves a device design. In an embodiment, the workflow (e.g., including timelines, and triggering events) is modified based on user-specific data to ensure manufacturing of user-specific device as desired and in compliance with regulatory specification, if any.

At operation 904, based on the received information, an interactive digital model associated with the device may be generated within the manufacturing workflow. For example, after the requesting entity submits the information via the graphical user interface, the system 100 may assign the request to a designing entity. The designing entity (an example of the second entity) may generate the interactive digital model based on the received patient-specific information and the workflow information. In an embodiment, generating the design of the patient-specific medical device involves determining a geometry of the patient-specific medical device based on the patient-specific information, and a material of the medical device compatible with the anatomy of the body part of the patient or the intended use of the medical device. In an embodiment, based on the device design, the project generation subsystem 116 may updated the workflow information to optimize the manufacturing process and ensure timely manufacturing and delivery of the medical device.

In an embodiment, determining the manufacturing process involves determining one or more manufacturing tools configured to manufacture geometric shape, density, surface finish and other properties of the patient-specific medical device. In an embodiment, the one or more manufacturing tools may be a three-dimensional (3D) printer configured to receive the design of the patient-specific medical device and print the patient-specific medical device using suitable material based on the intended use of the device; a tool step-up in a controlled manufacturing environment, a post-processing equipment configured for cleaning, curing, or finishing the medical device, or other manufacturing apparatus.

In some embodiment, operation 904 may be performed by a subsystem that is the same as or similar to the model generation subsystem 114 and the graphical user interface subsystem 120, in accordance with one or more embodiments.

At operation 906, the interactive digital model may be transmitted to the graphical user interface interaction with the requesting entity. In an embodiment, the operation 906 involves transmitting the interactive digital model of the patient-specific medical device to the graphical user interface (e.g., GUI 300/300A/300B). The graphical user interface may be configured to allow the requesting entity to interact with the interactive digital model.

In some embodiment, operation 906 may be performed by a subsystem and the cloud 150 that is the same as or similar to the graphical user interface subsystem 120, in accordance with one or more embodiments.

At operation 908, feedback from the requesting entity may be received on the interactive digital model. The feedback may include errors or modifications associated with the interactive digital model. In an embodiment, the feedback comprising errors or modifications associated with the patient-specific medical device. In an embodiment, the feedback may be a manipulation, an annotation, a highlight, and/or a comment associated with the interactive digital model.

In an embodiment, receiving the feedback from the requesting entity on the interactive digital model involves causing the graphical user interface to display and manipulate the interactive digital model of the patient-specific medical device, or an environment of the subject entity's anatomy adjacent to each other or superimposed on each other in a first portion of the graphical user interface; causing the graphical user interface to annotate and/or highlight one or more portions of the interactive digital model of the patient-specific medical device; and causing the graphical user interface to display editable comments box in a second portion of the graphical user interface, the second portion being adjacent to the first portion.

In some embodiment, operation 908 may be performed by a subsystem that is the same as or similar to the feedback subsystem 116 and the graphical user interface subsystem 120, in accordance with one or more embodiments.

In operation 910, updating, based on the feedback, the graphical user interface to display an updated interactive digital model associated with the device. In an embodiment, the operation 910 involves dynamically updating the graphical user interface to display an updated interactive digital model of the medical device based on the feedback from a healthcare profession. In an embodiment, the operation 910 involves transmitting the feedback on the interactive digital model associated with the patient-specific medical device to the designing entity; changing based on the feedback the design and corresponding interactive digital model associated with the user specific device by the designing entity, and/or the manufacturing process of the user-specific device; and reloading, on the graphical user interface, the updated interactive digital model for further comments or approval by the requesting entity.

In an embodiment, changing the interactive digital model associated with the patient-specific medical device involves analyzing the feedback to determine changes to the interactive digital model; and updating the resources required to incorporate the changes to the interactive digital model and/or the manufacturing process of the patient-specific medical device.

In an embodiment, the operation 910 involves displaying a 3D model of the patient-specific medical device and the structure of the body part adjacent to each other or superimposed on each other as a digital image in a 3D viewer; manipulating, by the requesting entity, the digital image to include or remove certain features of the device or the structure of the body part to better understand the structure of the body and/or an interaction of the patient-specific medical device with the structure of the body part; tagging or highlight one or more portions the digital image to bring them to the designing entity's attention; annotating, by the requesting entity, the digital image to provide feedback to the designing entity involved in the design, or a manufacturing entity involved in manufacturing of the patient-specific medical device; and/or receiving an approval of the digital image from the requesting entity and transmitting the approval to the designing entity or the manufacturing entity.

In some embodiment, operation 910 may be performed by a subsystem that is the same as or similar to the feedback subsystem 116 and the graphical user interface subsystem 120, in accordance with one or more embodiments.

At operation 912, once the updated interactive digital model is approved, the user-specific device (e.g., the patient-specific medical device) may be manufactured by a manufacturing entity. In an embodiment, the manufacturing entity is already ready to manufacture the updated user-specific device as the workflow was updated through the design and feedback process.

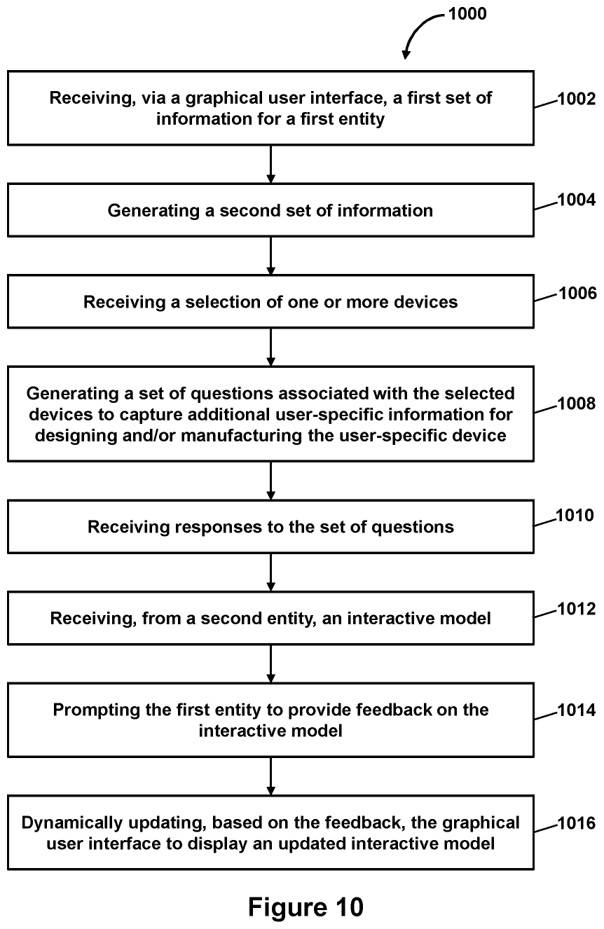

FIG. 10 illustrates another exemplary flowchart of a method 1000 for controlling a manufacturing process of a user-specific device (e.g., patient-specific medical device), according to an embodiment. Operation 1002 involves receiving, via a graphical user interface, a first set of information related to a user-specific device from a requesting entity, the first set of information comprises a type of application, structure of a body part, an intended use of the user-specific device, and intended outcome upon use of the user-specific device. The operation 1002 may be performed by a subsystem that is the same as or similar to the dynamic generation input subsystem 112 and the graphical user interface subsystem 120, in accordance with one or more embodiments.

Operation 1004 involves generating a second set of information based on the first set of information, the second set of information comprising a list of devices and device characteristics based on the first set of information related to the user-specific device. In an embodiment, the second set of information may be generated by determining, based on the selected one or more devices and the received information, suggested complementary or alternative device to the user-specific device selected by the requesting entity. The complementary or alternative device may be, for example, a surgical guide, an anatomical model made of bone or flesh like material complementing the user-specific device, a simulation model, or other devices.

The operation 1004 may be performed by a subsystem that is the same as or similar to the dynamic generation input subsystem 112 and the graphical user interface subsystem 120, in accordance with one or more embodiments.

Operation 1006 involves receiving, via the graphical user interface, a selection of one or more devices from the list of devices from the requesting entity. The operation 1006 may be performed by a subsystem that is the same as or similar to the dynamic generation input subsystem 112 and the graphical user interface subsystem 120, in accordance with one or more embodiments.

Operation 1008 involves generating a set of questions associated with the selected devices to capture additional user-specific information for designing and/or manufacturing the user-specific device. The operation 1008 may be performed by a subsystem that is the same as or similar to the dynamic generation input subsystem 112 and the graphical user interface subsystem 120, in accordance with one or more embodiments.

Operation 1010 involves receiving, via the graphical user interface, responses to the set of questions. The operation 1010 may be performed by a subsystem that is the same as or similar to the dynamic generation input subsystem 112 and the graphical user interface subsystem 120, in accordance with one or more embodiments.

In operation 1012 involves receiving, from a second entity, an interactive digital model associated with the user-specific device and/or a manufacturing process for manufacturing the user-specific device, the interactive digital model being generated based on the selected devices and responses to the set of questions. The operation 1012 may be performed by a subsystem that is the same as or similar to the model generation subsystem 114 and the graphical user interface subsystem 120, in accordance with one or more embodiments.

Operation 1014 involves prompting, via the graphical user interface, the requesting entity to provide feedback on the interactive digital model, the feedback comprising errors or modifications associated with the interactive digital model. The operation 1014 may be performed by a subsystem that is the same as or similar to the feedback subsystem 116 and the graphical user interface subsystem 120, in accordance with one or more embodiments.

Operation 1016 involves dynamically updating, based on the feedback, the graphical user interface to display an updated interactive digital model associated with the device for approval by the requesting entity. The operation 1016 may be performed by a subsystem that is the same as or similar to the feedback subsystem 116 and the graphical user interface subsystem 120, in accordance with one or more embodiments.

It can be understood that the systems and methods herein use medical devices as an example application to explain the concepts of the present disclosure. However, the scope of the present disclosure is not limited to medical devices. The technology platform herein can be used for controlling design and manufacturing processes associated with other user-specific devices such as mechanical devices, electrical devices, or electronic devices that have particular user-specific characteristics and may not be mass manufactured.

In some embodiments, the various computers and subsystems illustrated in FIG. 2 may include one or more computing devices that are programmed to perform the functions described herein. The computing devices may include one or more electronic storages (e.g., prediction database(s) 132, which may include training data database(s) 134, model database(s) 136, etc., or other electronic storages), one or more physical processors programmed with one or more computer program instructions, and/or other components. The computing devices may include communication lines or ports to enable the exchange of information within a network (e.g., network 150) or other computing platforms via wired or wireless techniques (e.g., Ethernet, fiber optics, coaxial cable, WiFi, Bluetooth, near field communication, or other technologies). The computing devices may include a plurality of hardware, software, and/or firmware components operating together. For example, the computing devices may be implemented by a cloud of computing platforms operating together as the computing devices.

The electronic storages may include non-transitory storage media that electronically stores information. The storage media of the electronic storages may include one or both of (i) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storage may store software algorithms, information determined by the processors, information obtained from servers, information obtained from client devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112-120 or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems 112-120 described herein is for illustrative purposes, and is not intended to be limiting, as any of subsystems 112-120 may provide more or less functionality than is described. For example, one or more of subsystems 112-120 may be eliminated, and some or all of its functionality may be provided by other ones of subsystems 112-120. As another example, additional subsystems may be programmed to perform some or all of the functionality attributed herein to one of subsystems 112-120.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

The present techniques will be better understood with reference to the following enumerated embodiments:

1. A method involves: receiving, via a graphical user interface, from a requesting entity, (i) information related to a device to be manufactured to conform to a subject entity, (ii) a selection by the requesting entity of one or more devices from a list of devices, where the information comprises: a type of application, structure of a body part of the subject entity, an intended use of a device, and intended outcome upon use of a device; generating, based on the received information and the selection of the one or more devices, a sequence of tasks for design and manufacturing of the user-specific device; generating an interactive digital model of the user-specific device, the interactive digital model being generated by a designing entity based on the received information and within the sequence of tasks generated by the system; transmitting the interactive digital model associated with the user-specific device to the graphical user interface, the graphical user interface being configured to allow the requesting entity to interact with the interactive digital model; receiving, via the graphical user interface, feedback from the requesting entity on the interactive digital model, the feedback comprising errors or modifications associated with the user-specific device; and updating, based on the feedback, the interactive digital model by the designing entity and the sequence of tasks to incorporate the feedback and design changes by the designing entity.

2. The method of embodiment 1, where the list of devices includes one or more of: an anatomical model of the part of the subject entity; a surgical guide to be used during surgery; or an implant.

3. The method of any of embodiments 1-2, where the information further comprises device characteristics is at least one of: a particular material based on the intended use of the device; a customized geometry to fit the structure of the subject entity's body part; and a sub-component or a particular area of interest of the structure of the subject entity's body part.

4. The method of embodiment 3, where the particular material for the device has one or more material property comprising: biocompatibility, flexibility, durability, transparency, utility and/or life-like appearance.

5. The method of any of embodiments 1-4, where the intended use of the device comprises at least one of: a pre- or an intra-surgical planning; visual communications; surgical simulation prior to an intended procedure; and an implantation in the subject entity.

6. The method of any of embodiments 1-5, where receiving the selection of the one or more devices comprises: determining the list of devices based on the type of application.

7. The method of any of embodiments 1-6, where receiving the list of devices comprises: determining, based on the selected one or more devices and the received information, suggested complementary or alternative devices to the user-specific device selected by the requesting entity.

8. The method of any of embodiments 1-7, where generating the sequence of tasks comprises: selecting, based on the information provided by the requesting entity, the sequence of tasks from a workflow database corresponding to the device characteristics, the intended use of the device, an acceptable quality specification, or regulation specifications associated with the user-specific device.

9. The method of any of embodiments 1-8, where generating the interactive digital model of the device comprises: determining, by the designing entity, a location and an orientation within the subject entity's environment, a geometry of the user-specific device based on the information provided by the requesting entity, and a material of the user-specific device compatible with the intended use of the device.

10. The method of any of embodiments 1-9, where determining the manufacturing process comprises: determining one or more manufacturing tools configured to manufacture a geometric shape, material, density, surface finish and other properties of the user-specific device.

11. The method of embodiment 10, where the one or more manufacturing tools comprises: a three-dimensional (3D) printer configured to receive the design of the user-specific device and 3D print the user-specific device using suitable material based on the intended use of the device; a controlled manufacturing environment, which allows particulate level and microbial counts to be kept below a safe threshold ensuring suitable cleanliness of the user specific device; and a post-processing equipment configured for cleaning, curing, or finishing the device.

12. The method of any of embodiments 1-11, further comprises: assigning of a unique identifier to the user-specific device; tracking, based on the unique identifier, changes to the interactive digital model or the device design associated with the user-specific device; tracking, based on the unique identifier, progress of one or more task within the sequence of tasks for the design and manufacture of the user-specific device; storing, based on the unique identifier, the design and user-specific data associated with the user-specific device; and identifying, based on the unique identifier, the manufactured user-specific device.

13. The method of any of embodiments 1-12, receiving the feedback from the requesting entity on the interactive digital model comprises: causing the graphical user interface to display and manipulate the interactive digital model of the user-specific device, or the environment of the subject entity adjacent to each other or superimposed on each other in a first portion of the graphical user interface; causing the graphical user interface to annotate and/or highlight one or more portions of the interactive digital model of the user-specific device; and causing the graphical user interface to display an editable comments box in a second portion of the graphical user interface, the second portion being adjacent to the first portion; and receiving, from the requesting entity, a manipulation, an annotation, a highlight, and/or, a comment associated with the interactive digital model.

14. The method of any of embodiments 1-13, receiving the feedback from the requesting entity on the interactive digital model comprises: receiving from the requesting entity a confirmation that the interactive digital model is approved; and transmitting that approval to the designing entity.

15. The method of any of embodiments 1-14, further comprises: transmitting the feedback on the interactive digital model associated with the user-specific device to the designing entity; changing based on the feedback, the design and corresponding interactive digital model associated with the user specific device by the designing entity, and/or the manufacturing process of the user-specific device; and reloading, on the graphical user interface, the updated interactive digital model for further comments or approval by the requesting entity.

16. The method of embodiment 15, where changing the interactive digital model associated with the user-specific device comprises: analyzing the feedback to determine changes to the interactive digital model; and updating resources required for the manufacturing process of the user-specific device.

17. The method of any of embodiments 1-16, further comprises: allocating, based on the information, and/or the feedback from the requesting entity, one or more resources for the design and/or manufacturing of the user-specific device based on capability and capacity of the one or more resources, the allocated resource comprising the designing entity and a manufacturing entity.

18. The method of embodiment 17, where allocating the one or more resources comprises: predicting an amount of time and a number of personnel required for designing and/or manufacturing of the user-specific device; predicting a total cost of the design and manufacture of the user-specific device; and determining, based on the time and cost, whether additional resources than the designing and manufacturing entity are required within the sequence of tasks to meet any deadline for supply of the user-specific device.

19. The method of embodiment 18, where allocating the one or more resources comprises: distributing of the device design, the interactive digital model or the updated interactive digital model associated with the user-specific device to one or more personnel across distributed sites; and/or distributing of the manufacturing of the user-specific device to one or more distributed sites.

20. The method of embodiment 19, where allocating the one or more resources comprises: assigning manufacturing resources based on a geographical location of the requesting entity, the capability and capacity associated with the one or more available resources of the designing and manufacturing entities.

21. The method of any of embodiments 1-20, further comprises: receiving a geographical location as part of the information received from the requesting entity; determining, based on the geographical location, a relevant jurisdiction associated with the geographical location, regulatory specifications and/or quality assurance constraints related to a user-specific device for that jurisdiction; updating the manufacturing workflow for the designing and manufacturing entities to comply with the requirements of the jurisdiction; and generating regulatory and quality assurance documentations to meet the requirements of the jurisdiction.

22. A method for dynamically updating a graphical user interface for controlling design and manufacturing processes for user-specific devices, the method involving: receiving, via a graphical user interface, a first set of information related to a user-specific device from a requesting entity, the first set of information comprises a type of application, structure of a body part, an intended use of the user-specific device, and intended outcome upon use of the user-specific device; generating a second set of information based on the first set of information, the second set of information comprising a list of devices and device characteristics based on the first set of information related to the user-specific device; receiving, via the graphical user interface, a selection of one or more devices from the list of devices from the requesting entity; generating a set of questions associated with the selected devices to capture additional user-specific information for designing and/or manufacturing the user-specific device; receiving, via the graphical user interface, responses to the set of questions; receiving, from a designing entity, an interactive digital model associated with the user-specific device and/or a manufacturing process for manufacturing the user-specific device, the interactive digital model being generated based on the selected devices and responses to the set of questions; prompting, via the graphical user interface, the requesting entity to provide feedback on the interactive digital model, the feedback comprising errors or modifications associated with the interactive digital model; and updating, based on the feedback, the graphical user interface to display an updated interactive digital model associated with the device for approval by the requesting entity.

23. A method for controlling design and manufacturing processes of a medical device for a patient, the method involves: receiving, via a graphical user interface, patient-specific information related to a medical procedure to be performed by a requesting entity, the patient-specific information comprises a type of application, anatomy of a subject entity, an intended use of the medical device, and intended outcome upon use of the medical device; determining a list of medical devices and device characteristics based on the patient-specific information related to the medical procedure; responsive to selection of one or more medical devices from the list of medical devices by the requesting entity, determining additional patient-specific information to be requested from the requesting entity; generating, based on the received information and the selection of the one or more devices, a sequence of tasks for design and manufacturing of the patient-specific device; generating an interactive digital model for a patient-specific medical device and/or a manufacturing process for manufacturing the patient-specific medical device, the interactive digital model being generated by a designing entity based on the received information and the sequence of tasks; transmitting the interactive digital model of the patient-specific medical device to the graphical user interface, the graphical user interface being configured to allow the requesting entity to interact with the interactive digital model; receiving, via the graphical user interface, feedback from the requesting entity on the interactive digital model, the feedback comprising errors or modifications associated with the patient-specific medical device; and updating, based on the feedback, the interactive digital model by the designing entity and the sequence of tasks to incorporate the feedback and design changes by the designing entity.

24. The method of embodiments 23, where the list of medical device comprises one or more of: an anatomical model of a part of the subject entity; a surgical guide to be used during surgery; or an implant.

25. The method of any of embodiments 23-24, where the device characteristics is at least one of: a particular material based on the intended use of the patient-specific medical device;

a customized geometry to fit the anatomy of the subject entity's body part during a particular part of the medical procedure; and a sub-component or a particular area of interest of the structure of the subject entity's anatomy.

26. The method of embodiment 25, where the particular material for the medical device has one or more material property comprising: biocompatibility, flexibility, durability, transparency, utility and/or life-like appearance.

27. The method of any of embodiments 23-26, where the intended use of the medical device comprises at least one of: a pre- or an intra-surgical planning; visual communication; surgical simulation prior to the intended procedure; and an implantation in the subject entity's body part.

28. The method of embodiment 27, where receiving the selection of the one or more devices comprises: receiving a selection from the list of devices based on the type of application.

29. The method of any of embodiments 23-28, where determining the list of devices comprises:

determining, based on the selected one or more devices and the received information, suggested complementary or alternative device to the patient-specific device selected by the requesting entity.

30. The method of any of embodiments 23-29, where generating the sequence of tasks comprises: selecting, based on the information provided by the requesting entity, the sequence of tasks from a workflow database corresponding to the medical device characteristics, the intended use of the medical device, an acceptable quality specification, or regulation specifications associated with the user-specific device.

31. The method of any of embodiments 23-30, where generating the interactive digital model of the patient-specific medical device comprises: determining, by the designing entity, a location and an orientation within the subject entity's anatomy, a geometry of the patient-specific medical device based on the information provided by the requesting entity, and a material of the patient-specific medical device compatible with the intended use of the device.

32. The method of any of embodiments 23-31, where determining the manufacturing process comprises: determining one or more manufacturing tools configured to manufacture a geometric shape, material, density, surface finish and/or other properties of the user-specific device.

33. The method of embodiment 32, where the one or more manufacturing tools comprises: a three-dimensional (3D) printer configured to receive the design of the patient-specific medical device and print the patient-specific medical device using suitable material based on the intended use of the patient-specific medical device; a controlled manufacturing environment, which allows particulate level and microbial counts to be kept below a safe threshold ensuring suitable cleanliness of the patient-specific medical device; and a post-processing equipment configured for cleaning, curing, or finishing the device.

34. The method of any of embodiments 23-33, receiving the feedback from the requesting entity on the interactive digital model comprises: causing the graphical user interface to display and manipulate the interactive digital model of the patient-specific medical device, or the anatomy of the subject entity adjacent to each other or superimposed on each other in a first portion of the graphical user interface; causing the graphical user interface to annotate and/or highlight one or more portions of the interactive digital model; causing the graphical user interface to display an editable comments box in a second portion of the graphical user interface, the second portion being adjacent to the first portion; and receiving, from the first requesting entity, a manipulation, an annotation, a highlight, and/or, a comment associated with the interactive digital model.

35. The method of any of embodiments 23-34, further involves: transmitting the feedback on the interactive digital model associated with the patient-specific medical device to the designing entity; and changing, based on the feedback, the interactive digital model associated with the patient-specific medical device by the designing entity and/or the manufacturing process of the patient-specific medical device.

36. The method of embodiment 35, where changing the interactive digital model associated with the patient-specific medical device comprises: analyzing the feedback to determine changes to the interactive digital model; and updating resources required for the manufacturing process of the patient-specific medical device.

37. The method of embodiment 34, further involves: allocating, based on the information, and/or the feedback from the requesting entity, one or more resources for the design and/or manufacturing of the patient-specific medical device based on capability and capacity of the one or more resources, the allocated resource comprising the designing entity and a manufacturing entity

38. The method of embodiment 37, where allocating the one or more resources comprises: predicting an amount of time and a number of personnel required for designing and/or manufacturing of the patient-specific medical device; predicting a total cost of the design and manufacture of the patient-specific medical device; and determining, based on the time and cost, whether additional resources than the designing and manufacturing entity are required to meet any deadline for supply of the user-specific device.

39. The method of embodiment 37, where allocating the one or more resources comprises: distributing of the device design, the interactive digital model or the updated interactive digital model associated with the patient-specific medical device to one or more personnel across distributed sites; and/or distributing of the manufacturing of the patient-specific medical device to one or more distributed sites.

40. The method of embodiment 37, where allocating the one or more resources comprises: assigning manufacturing resources to the sequence of tasks based on the geographical location of the requesting entity, the capability and capacity the one or more available resources of the designing and manufacturing entities.

41. The method of any of embodiments 23-40, further involves: receiving a geographical location as part of the information received from the requesting entity; determining, based on the geographical location, relevant jurisdiction associated with the geographical location, regulatory specifications and/or quality assurance constraints related to a patient-specific medical device for that jurisdiction; updating the manufacturing workflow for the designing and manufacturing entities to comply with the requirements of the jurisdiction; and generating regulatory and quality assurance documentations to meet the requirements of the jurisdiction.

42. A tangible, non-transitory, machine-readable medium storing instructions that, when executed by a data processing apparatus, causes the data processing apparatus to perform operations comprising those of any of embodiments of the methods above.

43. A system comprising: one or more processors; and memory storing instructions that, when executed by the processors, cause the processors to effectuate operations comprising those of any of embodiments of the methods above.

1. A system for controlling design and manufacturing processes for user-specific devices, the system comprising:
a computer system that comprises one or more processors programmed with computer program instructions that, when executed, cause the one or more processors to:
receive, via a graphical user interface, from a requesting entity, (i) information related to a device to be manufactured to conform to a subject entity, (ii) a selection by the requesting entity of one or more devices from a list of devices, wherein the information comprises: a type of application, structure of a body part of the subject entity, an intended use of a device, and intended outcome upon use of a device;
generate, based on the received information and the selection of the one or more devices, a sequence of tasks for design and manufacturing of the user-specific device;
generate an interactive digital model of the user-specific device, the interactive digital model being generated by a designing entity based on the received information and within the sequence of tasks generated by the system;
transmit the interactive digital model associated with the user-specific device to the graphical user interface, the graphical user interface being configured to allow the requesting entity to interact with the interactive digital model;
receive, via the graphical user interface, feedback from the requesting entity on the interactive digital model, the feedback comprising errors or modifications associated with the user-specific device; and
update, based on the feedback, the interactive digital model by the designing entity and the sequence of tasks to incorporate the feedback and design changes by the designing entity.

2. The system of claim 1, wherein the list of devices includes one or more of:
an anatomical model of the part of the subject entity;
a surgical guide to be used during surgery; or
an implant.

3. The system of claim 1 or 2, wherein the information further comprises device characteristics selected from at least one of:
a particular material based on the intended use of the device;
a customized geometry to fit a structure of the subject entity's body part; and
a sub-component or a particular area of interest of the structure of the subject entity's body part.

4. The system of claim 3, wherein the particular material for the device has one or more material property comprising: biocompatibility, flexibility, durability, transparency, utility and/or life-like appearance.

5. The system of claim 1, wherein the intended use of the device comprises at least one of:
a pre- or an intra-surgical planning;
visual communications;
surgical simulation prior to an intended procedure; and
an implantation in the subject entity.

6. The system of claim 1, wherein to receive the selection of the one or more devices, the one or more processors are further caused to: determine the list of devices based on the type of application.

7. The system of claim 1, wherein to receive the list of devices, the one or more processors are further caused to:
determine, based on the selected one or more devices and the received information, suggested complementary or alternative devices to the user-specific device selected by the requesting entity.

8. The system of claim 3, wherein to generate the sequence of tasks, the one or more processors are further caused to:
select, based on the information provided by the requesting entity, the sequence of tasks from a workflow database corresponding to the device characteristics, the intended use of the device, an acceptable quality specification, or regulation specifications associated with the user-specific device.

9. The system of claim 1, wherein to generate the interactive digital model of the device, the one or more processors are further caused to:
determine, by the designing entity, a location and an orientation within the subject entity's environment, a geometry of the user-specific device based on the information provided by the requesting entity, and a material of the user-specific device compatible with the intended use of the device.

10. The system of claim 1, wherein to determine the manufacturing process, the one or more processors are further caused to:
determine one or more manufacturing tools configured to manufacture a geometric shape, material, density, and/or surface finish of the user-specific device.

11. The system of claim 10, wherein the one or more manufacturing tools comprises:
a three-dimensional (3D) printer configured to receive the design of the user-specific device and 3D print the user-specific device using suitable material based on the intended use of the device;
a controlled manufacturing environment, which allows particulate level and microbial counts to be kept below a safe threshold ensuring suitable cleanliness of the user-specific device; and
a post-processing equipment configured to clean, cure, or finish the device.

12. The system of claim 1, wherein the one or more processors are further caused to:
assign a unique identifier to the user-specific device;
track, based on the unique identifier, changes to the interactive digital model or the device design associated with the user-specific device;
track, based on the unique identifier, progress of one or more task within the sequence of tasks for the design and manufacture of the user-specific device,
store, based on the unique identifier, the design and user-specific data associated with the user-specific device; and
identify, based on the unique identifier, the manufactured user-specific device.

13. The system of claim 1, wherein to receive the feedback from the requesting entity on the interactive digital model, the one or more processors are further caused to:
cause the graphical user interface to display and manipulate the interactive digital model of the user-specific device, or the environment of the subject entity adjacent to each other or superimposed on each other in a first portion of the graphical user interface;
cause the graphical user interface to annotate and/or highlight one or more portions of the interactive digital model of the user-specific device;
cause the graphical user interface to display an editable comments box in a second portion of the graphical user interface, the second portion being adjacent to the first portion; and
receive, from the requesting entity, a manipulation, an annotation, a highlight, and/or, a comment associated with the interactive digital model.

14. The system of claim 1, wherein to receive the feedback from the requesting entity on the interactive digital model, the one or more processors are further caused to: receive from the requesting entity a confirmation that the interactive digital model is approved; and transmitting that approval to the designing entity.

15. The system of claim 1, further comprises:
transmit the feedback on the interactive digital model associated with the user-specific device to the designing entity;
change based on the feedback, the design and corresponding interactive digital model associated with the user specific device by the designing entity, and/or the manufacturing process of the user-specific device; and
reload, on the graphical user interface, the updated interactive digital model for further comments or approval by the requesting entity.

16. The system of claim 15, wherein to change the interactive digital model associated with the user-specific device, the one or more processors are further caused to:
analyze the feedback to determine changes to the interactive digital model; and
update resources suggested for the manufacturing process of the user-specific device.

17. The system of claim 1, wherein the one or more processors are further caused to:
allocate, based on the information, and/or the feedback from the requesting entity, one or more resources for the design and/or manufacturing of the user-specific device based on capability and capacity of the one or more resources, the allocated resource comprising the designing entity and a manufacturing entity.

18. The system of claim 17, wherein to allocate the one or more resources, the one or more processors are further caused to:
predict an amount of time and a number of personnel required for designing and/or manufacturing of the user-specific device;
predict a total cost of the design and manufacture of the user-specific device; and
determine, based on the time and cost, whether additional resources than the designing and manufacturing entity are required within the sequence of tasks to meet any deadline for supply of the user-specific device.

19. The system of claim 18, wherein to allocate the one or more resources, the one or more processors are further caused to:
distribute of the device design, the interactive digital model or the updated interactive digital model associated with the user-specific device to one or more personnel across distributed sites; and/or
distribute of the manufacturing of the user-specific device to one or more distributed sites.

20. The system of claim 19, wherein to allocate the one or more resources, the one or more processors are further caused to:
assign manufacturing resources based on a geographical location of the requesting entity, the capability and capacity associated with one or more available resources of the designing and manufacturing entities.

21. The system of claim 1, wherein the one or more processors are further caused to:
receive a geographical location as part of the information received from the requesting entity;
determine, based on the geographical location, relevant jurisdiction associated with the geographical location, regulatory specifications and/or quality assurance constraints related to a user-specific device for that jurisdiction;
update the sequence of tasks for the designing and manufacturing entities to comply with the requirements of the jurisdiction; and
generate regulatory and quality assurance documentations to meet the requirements of the jurisdiction.

22. A computer-implemented method for dynamically updating a graphical user interface for controlling design and manufacturing processes for user-specific devices, the method comprising:
receiving, via a graphical user interface, a first set of information related to a user-specific device from a requesting entity, the first set of information comprises a type of application, structure of a body part, an intended use of the user-specific device, and intended outcome upon use of the user-specific device;
generating a second set of information based on the first set of information, the second set of information comprising a list of devices and device characteristics based on the first set of information related to the user-specific device;
receiving, via the graphical user interface, a selection of one or more devices from the list of devices from the requesting entity;
generating a set of questions associated with the selected devices to capture additional user-specific information for designing and/or manufacturing the user-specific device;
receiving, via the graphical user interface, responses to the set of questions;
receiving, from a designing entity, an interactive digital model associated with the user-specific device and/or a manufacturing process for manufacturing the user-specific device, the interactive digital model being generated based on the selected devices and responses to the set of questions;
prompting, via the graphical user interface, the requesting entity to provide feedback on the interactive digital model, the feedback comprising errors or modifications associated with the interactive digital model; and
updating, based on the feedback, the graphical user interface to display an updated interactive digital model associated with the device for approval by the requesting entity.

23. A system for controlling design and manufacturing processes of a medical device for a patient, the system comprising:
a computer system that comprises one or more processors programmed with computer program instructions that, when executed, cause the one or more processors to:
receive, via a graphical user interface, patient-specific information related to a medical procedure to be performed by a requesting entity, the patient-specific information comprises a type of application, anatomy of a subject entity, an intended use of the medical device, and intended outcome upon use of the medical device;
determine a list of medical devices and device characteristics based on the patient-specific information related to the medical procedure;
responsive to selection of one or more medical devices from the list of medical devices by the requesting entity, determining additional patient-specific information to be requested from the requesting entity;
generate, based on the received information and the selection of the one or more devices, a sequence of tasks for design and manufacturing of the patient-specific device;
generate, an interactive digital model for the patient-specific medical device and/or a manufacturing process for manufacturing the patient-specific medical device, the interactive digital model being generated by a designing entity based on the received information and the sequence of tasks;
transmit the interactive digital model of the patient-specific medical device to the graphical user interface, the graphical user interface being configured to allow the requesting entity to interact with the interactive digital model;
receive, via the graphical user interface, feedback from the requesting entity on the interactive digital model, the feedback comprising errors or modifications associated with the patient-specific medical device; and
update, based on the feedback, the interactive digital model by the designing entity and the sequence of tasks to incorporate the feedback and design changes by the designing entity.

24. The system of claim 23, wherein the list of medical device comprises one or more of: an anatomical model of a part of the subject entity;
a surgical guide to be used during surgery; or
an implant.

25. The system of claim 23, wherein the device characteristics is at least one of:
a particular material based on the intended use of the patient-specific medical device;
a customized geometry to fit the anatomy of the subject entity during a particular part of the medical procedure; and
a sub-component or a particular area of interest of a structure of the subject entity's anatomy.

26. The system of claim 25, wherein the particular material for the medical device has one or more material property comprising: biocompatibility, flexibility, durability, transparency, utility and/or life-like appearance.

27. The system of claim 23, wherein the intended use of the medical device comprises at least one of:
a pre- or an intra-surgical planning;
visual communication;
surgical simulation prior to the intended procedure; and
an implantation in a body part of the subject entity.

28. The system of claim 27, wherein to receive the selection of the one or more devices, the one ore more processors are further caused to:
receive a selection from the list of devices based on the type of application.

29. The system of claim 23, wherein to determine the list of devices, the one or more processors are further caused to:
determining, based on the selected one or more devices and the received information, suggested complementary or alternative device to the patient-specific device selected by the requesting entity.

30. The system of claim 23, wherein to generate the sequence of tasks, the one or more processors are further caused to:
select, based on the information provided by the requesting entity, the sequence of tasks from a workflow database corresponding to the medical device characteristics, the intended use of the medical device, an acceptable quality specification, or regulation specifications associated with the patient-specific medical device.

31. The system of claim 23, wherein to generate the interactive digital model of the patient-specific medical device, the one or more processors are further caused to:
determine, by the designing entity, a location and an orientation within the subject entity's anatomy, a geometry of the patient-specific medical device based on the information provided by the requesting entity, and a material of the patient-specific medical device compatible with the intended use of the device.

32. The system of claim 23, wherein to determine the manufacturing process comprises:
determining one or more manufacturing tools configured to manufacture a geometric shape, material, density, and/or surface finish of the patient-specific medical device.

33. The system of claim 32, wherein the one or more manufacturing tools comprises:
a three-dimensional (3D) printer configured to receive the design of the patient-specific medical device and print the patient-specific medical device using suitable material based on the intended use of the patient-specific medical device;
a controlled manufacturing environment, which allows particulate level and microbial counts to be kept below a safe threshold ensuring suitable cleanliness of the patient-specific medical device; and
a post-processing equipment configured to clean, cure, or finish the device.

34. The system of claim 23, wherein to receive the feedback from the requesting entity on the interactive digital model, the one or more processors are further caused to:
cause the graphical user interface to display and manipulate the interactive digital model of the patient-specific medical device, or the anatomy of the subject entity adjacent to each other or superimposed on each other in a first portion of the graphical user interface;
cause the graphical user interface to annotate and/or highlight one or more portions of the interactive digital model;
cause the graphical user interface to display an editable comments box in a second portion of the graphical user interface, the second portion being adjacent to the first portion; and
receive, from the first requesting entity, a manipulation, an annotation, a highlight, and/or, a comment associated with the interactive digital model.

35. The system of claim 23, wherein the one or more processors are further caused to:
transmit the feedback on the interactive digital model associated with the patient-specific medical device to the designing entity; and
change, based on the feedback, the interactive digital model associated with the patient-specific medical device by the designing entity and/or the manufacturing process of the patient-specific medical device.

36. The system of claim 35, wherein to change the interactive digital model associated with the patient-specific medical device, the one or more processors are further caused to:
analyze the feedback to determine changes to the interactive digital model; and
update resources required for the manufacturing process of the patient-specific medical device.

37. The system of claim 34, the one or more processors are further caused to:
allocate, based on the information, and/or the feedback from the requesting entity, one or more resources for the design and/or manufacturing of the patient-specific medical device based on capability and capacity of the one or more resources, the allocated resource comprising the designing entity and a manufacturing entity

38. The system of claim 37, wherein to allocate the one or more resources, the one or more processors are further caused to:
predict an amount of time and a number of personnel required for designing and/or manufacturing of the patient-specific medical device,
predict a total cost of the design and manufacture of the patient-specific medical device; and
determine, based on the time and cost, whether additional resources than the designing and manufacturing entity are required to meet any deadline for the supply of the patient-specific medical device.

39. The system of claim 37, wherein to allocate the one or more resources, the one or more processors are further caused to:
distribute device design, the interactive digital model or the updated interactive digital model associated with the patient-specific medical device to one or more personnel across distributed sites; and/or
distributed manufacturing of the patient-specific medical device to one or more distributed sites.

40. The system of claim 37, wherein to allocate the one or more resources, the one or more processors are further caused to:
assign manufacturing resources to the manufacturing workflow based on a geographical location of the requesting entity, the capability and capacity associated with one or more available resources of the designing and manufacturing entities.

41. The system of claim 1, further comprises:
receive a geographical location as part of the information received from the requesting entity;
determine, based on the geographical location, relevant jurisdiction associated with the geographical location, regulatory specifications and/or quality assurance constraints related to a patient-specific medical device for that jurisdiction;
update the manufacturing workflow for the designing and manufacturing entities to comply with the requirements of the jurisdiction; and
generate regulatory and quality assurance documentations to meet the requirements of the jurisdiction.
